(19) United States
(12) Patent Application Publication
(10) Pub. No.: US 2021/0205236 A1
(43) Pub. Date: Jul. 8, 2021

(54) THERAPEUTIC COMBINATIONS OF BOSWELLIA EXTRACT AND CANNABINOIDS

(71) Applicant: EMERALD HEALTH THERAPEUTICS CANADA INC., Vancouver (CA)

(72) Inventors: Freydoun GARABAGI, Vancouver (CA); Nancy E. HARRISON, Vancouver (CA); Salam A. KADHIM, Vancouver (CA); Christopher WAGNER, Vancouver (CA)

(21) Appl. No.: 17/271,150

(22) PCT Filed: Aug. 26, 2019
(86) PCT No.: PCT/IB2019/000977

§ 371 Date: Feb. 24, 2021

Related U.S. Application Data
(60) Provisional application No. 62/723,260, filed on Aug. 27, 2018.

(51) Int. Cl. A61K 31/05; A61K 36/324; A61K 9/48; A61K 9/00; A61K 9/20

(52) U.S. Cl. CPC A61K 31/05; A61K 36/324; A61K 9/4858; A61K 31/352; A61K 9/0095; A61K 9/20; A61K 9/0056

(57) ABSTRACT

Described herein are cannabinoid formulations in combination with Boswellia extract for oral administration. Further described herein are methods for orally administering one or more cannabinoids to a subject in need thereof and manufacturing oral formulations as described herein.

### CROSS-REFERENCE TO RELATED APPLICATIONS

This application claims the benefit of U.S. Provisional Application No. 62/723,260, filed Aug. 27, 2018, which is incorporated herein by reference in its entirety.

### BACKGROUND OF THE INVENTION

Natural health products (NHPs), also known as dietary supplements, are manufactured products intended to supplement the diet when taken by mouth as a pill, capsule, tablet, or liquid. NHPs provide nutrients either extracted from food sources, animals or synthetically made, in order to restore or maintain good health and to increase the quantity of their consumption in the normal diet or because the nutrient is not available in a regular diet. NHPs are widely available. It is estimated that over 50% of North American citizens regularly consume one or more NHPs such as vitamins, amino acids, plants or plant extracts. In the United States and Canada, NHPs and dietary supplements are considered a subset of foods and are regulated accordingly. The European Commission has also established harmonized rules to ensure that NHPs are safe and properly labeled.

Cannabinoids from the plant genus Cannabis could be considered a type of natural health product, but historically they have not been legally available. The laws which have criminalized possession or use of Cannabis have been the primary restraint. These laws were put in place apparently to control the use of one specific cannabinoid, delta-9 tetrahydrocannabinol (THC), which causes a mild temporary psychotropic effect in users. But it is well known that dozens of other cannabinoids are also present in Cannabis, none of which have psychotropic effects, and which have, or potentially may have, beneficial pharmacological effects in humans. These alternate cannabinoids which are devoid of psychotropic effect include but are not limited to tetrahydrocannabinolic acid (THCA), cannabinolic acid (CBNA), cannabidiolic acid (CBDA) and cannabigerolic acid (CBGA) and the de-carboxylated derivatives cannabinol (CBN), cannabichromene (CBC), cannabidiol (CBD), cannabichromene (CBC) and cannabigerol (CBG). With the de-criminalization of Cannabis in some jurisdictions, the opportunity for use of cannabinoids in diverse health regimens is becoming possible.

Cannabinoids bind to receptors in the body known as cannabinoid receptors which have been implicated in a variety of physiological functions, including appetite, pain, emotional behavior (mood), memory, and inflammation. There are currently two known well defined subtypes of cannabinoid receptors. The CB₁ receptor (CB1R) is expressed mainly in the brain (central nervous system or “CNS”), and also in the lungs, liver and kidneys. The CB₂ receptor (CB2R) is expressed mainly in the immune system and in hematopoietic or blood cells. There is mounting evidence that there are other novel cannabinoid receptors which are similar to CB1R and CB2R but which are unique.

The potential to use cannabinoids and NHPs in combination has yet to be fully explored. The invention herein is directed to novel combinations comprising A) one or more NHPs, and B) one or more cannabinoids, in oral formulation. Such products are useful as natural health products, dietary supplements, and for treatment of human diseases, conditions and disorders.

### SUMMARY OF THE INVENTION

Provided herein are cannabinoid and Boswellia extract formulations, in combination, for oral administration.

In one aspect, described herein is an oral formulation comprising one or more cannabinoids selected from among the group consisting of: 0.1-750 mg tetrahydrocannabinolic acid (THCA), 0.1-100 mg tetrahydrocannabinol (THC), 0.1-750 mg cannabidiolic acid (CBDA), 0.1-750 mg cannabidiol (CBD), 0.1-750 mg cannabichromene (CBC), and 0.1-750 mg cannabigerol (CBG); and a Boswellia extract comprising 3-O-acetyl-11-ketobeta-boswellic acid (AKBA). In some embodiments, oral formulations are in a unit dosage form selected from the group consisting of a pill, tablet, capsule, film, wafer, lollipop, lozenge, oil, tincture, and syrup. In some embodiments, the formulation is an orally disintegrating pill, tablet, capsule, film, or wafer. In some embodiments, the formulation is a pill or tablet and further comprises an enteric coating for containing the one or more cannabinoids and the lipid carrier. In some embodiments, the formulation is a pill, tablet, or capsule, and further comprises an outer shell that is substantially opaque to one or both of ultraviolet and visible light. In some embodiments, the formulation further comprises a carrier oil. In some embodiments, the formulation further comprises a stabilizer. In some embodiments, one or more of the cannabinoids is present in the form of an organic solvent-based extract of Cannabis. In some embodiments, at least one further cannabinoid selected from the group consisting of CBGA, CBC, and THCV. In some embodiments, the formulation comprises CBD in an amount between 10-50 mg. In some embodiments, the formulation comprises 25 mg CBD. In some embodiments, the formulation comprises 500 mg CBD. In some embodiments, the Boswellia extract is or comprises a Boswellia serrata extract. In some embodiments, the Boswellia serrata extract is present in an amount of about 100 mg to 1000 mg, or about 250 mg, about 450 mg, or about 1000 mg. In some embodiments, the AKBA or the Boswellia extract, or both, is/are present in the form of an organic solvent-based extract. In some embodiments, the cannabinoid is physically separated from the Boswellia extract and/or the AKBA. In some embodiments, the cannabinoid is evenly dispersed within at least a portion of the oral formulation. In some embodiments, a signifier which signifies the cannabinoid dosage is associated directly with the oral formulation by embossing, or by colour, pattern or shape feature. In some embodiments, the signifier is adapted to be directly interpreted by a consumer and/or is a machine-readable code. In some embodiments, the oral formulation is contained in an individual blister pack sealed in an inert gas atmosphere comprising little or no oxygen. In some embodiments, the Boswellia serrata extract is standardized to 40% boswellic acid or AKBA.

In one aspect, described herein is a method of changing the level of a biomarker selected from IL-6, TNF-α, and C-reactive protein in the serum of an individual, the method comprising administering to the individual an oral formulation as described herein. In some embodiments, the individual is suffering from one or more diseases, conditions, or disorders selected from the group consisting of pain, inflammation, anxiety, depression, sleep disorders, insomnia, lack of energy, lack of alertness, weight gain, obesity, diabetes, Metabolic Syndrome, acute and anticipatory nausea, suppressed appetite, epilepsy, spasticity, schizophrenia, bi-polar disorder, cancer and neoplasia, chronic pain, osteoarthritic pain, and fibromyalgia. In some embodiments, the administering results in amelioration and/or treatment of one or more symptoms selected from the group consisting of pain, inflammation, anxiety, depression, sleep disorders, insomnia, lack of energy, lack of alertness, weight gain, obesity, diabetes, Metabolic Syndrome, acute and anticipatory nausea, suppressed appetite, epilepsy, spasticity, schizophrenia, bi-polar disorder, cancer and neoplasia, chronic pain, osteoarthritic pain, bacterial and/or fungal infections and fibromyalgia.

In one aspect, described herein is a method of treating an individual suffering from one or more diseases, conditions or disorders selected from the group consisting of pain, inflammation, anxiety, depression, sleep disorders, insomnia, lack of energy, lack of alertness, weight gain, obesity, diabetes, Metabolic Syndrome, acute and anticipatory nausea, suppressed appetite, epilepsy, spasticity, schizophrenia, bi-polar disorder, cancer and neoplasia, chronic pain, osteoarthritic pain, bacterial and/or fungal infections and fibromyalgia, the method comprising administration to the individual of a therapeutically effective amount of an oral formulation as described herein. In some embodiments, the disease or condition is associated with IL-6, TNF-α, or C-reactive protein. In some embodiments, the treating results in a change in a level of IL-6, TNF-α, or C-reactive protein in the serum of an individual. In some embodiments, the change is a reduction or an increase.

In one aspect, described herein is a method of manufacturing an oral formulation as described herein, comprising providing an organic extract of cannabinoids from cultivated Cannabis, measuring the concentration of one or more cannabinoids selected from the group consisting of THCA, THC, CBDA, and CBD in the organic extract, adjusting the concentration of one or more cannabinoids in the extract to prepare an adjusted extract within the concentration tolerance limits of a manufacturing specification for the oral formulation; and manufacturing the oral formulation with the adjusted extract.

In some embodiments, the formulation comprises a unit dose or combination dose of cannabinoid(s) selected from the list consisting of (each cannabinoid milligram amount about or equal to):
THC (10 mg), CBD (10 mg), and Boswellia extract (100 mg),
THC (10 mg), CBG (3 mg), and Boswellia extract (100 mg),
THC (1 mg), CBD (25 mg), and Boswellia extract (100 mg),
THC (10 mg), CBD (10 mg), and Boswellia extract (100 mg),
THC (5 mg), CBD (20 mg), and Boswellia extract (100 mg),
THC (10 mg), CBG (3 mg), and Boswellia extract (100 mg),
THC (1 mg), CBD (10 mg), and Boswellia extract (100 mg),
THC (1 mg), THCA (9 mg), and Boswellia extract (100 mg),
THC (10 mg), CBD (10 mg), and Boswellia extract (100 mg),
THC (10 mg) and Boswellia extract (180 mg),
THC (10 mg), CBD (10 mg), and Boswellia extract (200 mg),
THC (10 mg), CBG (3 mg), and Boswellia extract (100 mg),
THC (10 mg), CBD (10 mg), and Boswellia extract (40 mg),
THC (1 mg), CBD (10 mg), and Boswellia extract (40 mg),
THC (2.5 mg) and Boswellia extract (25 mg),
THC (5 mg) and Boswellia extract (50 mg),
THC (10 mg) and Boswellia extract (100 mg),
THC (10 mg), CBD (10 mg), and Boswellia extract (80 mg),
THC (1 mg), CBD (10 mg), and Boswellia extract (80 mg),
THC (1 mg), CBD (10 mg), and Boswellia extract (100 mg),
THCV (10 mg), CBD (10 mg), and Boswellia extract (40 mg),
THC (5 mg), CBD (10 mg), and Boswellia extract (100 mg),
THC (1 mg), CBD (10 mg), and Boswellia extract (200 mg),
THC (10 mg), CBC (10 mg), CBD (10 mg), and Boswellia extract (40 mg),
THC (10 mg), CBG (3 mg), CBC (3mg) and Boswellia extract (100 mg),
THC (10 mg), CBG (10 mg), CBC (10mg) and Boswellia extract (200 mg), and
THC (10 mg), CBG (3 mg), CBC (3mg) and Boswellia extract (200 mg).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 Depicts interactions between Boswellic Acid and human metabolic pathways.

FIG. 2. AKBA alone and AKBA combinations—Analysis of CB1R agonistic activity. A. Dose-response curve representation (log [AKBA concentration] (M) vs activity (Fold change)). Results are expressed as mean±S.E.M. (n=3). The EC50 value of AKBA-THC combination was determined by fitting a dose response curve with nonlinear regression log [agonist] (M) vs response (Fold change) using Prism 8 (GraphPad Software). B. Bar representation of CB1R activity (Fold change) of AKBA alone vs AKBA-THC combo treatments. Results are expressed as mean±S.E.M. (n=3). Statistical significance was determined by non-parametric Kruskal-Wallis test—Uncorrected Dunn's multiple comparisons test. *, p<0.05.

FIG. 3. AKBA alone and AKBA combinations—Comparison analysis of CB1R agonistic activity. A. Bar representation of CB1R activity (Fold change) of AKBA alone vs AKBA-THCA combo treatments. B. Bar representation of CB1R activity (Fold change) of AKBA alone vs AKBA-THC combo treatments. Results are expressed as mean±S.E.M. (n=3). Statistical significance was determined by non-parametric Kruskal-Wallis test—Uncorrected Dunn's multiple comparisons test. *, p<0.05. **, p<0.01.

FIG. 4. AKBA alone and AKBA combinations—Analysis of CB1R positive allosteric activity. A. Dose-response curve representation (log [AKBA concentration] (M) vs activity (Fold change)). Results are expressed as mean±S.E.M. (n=3). The EC50 values were determined by fitting a dose response curve with nonlinear regression log [agonist] (M) vs response (Fold change) using Prism 8 (GraphPad Software). B. Bar representation of CB1R positive allosteric activity EC50 values. Results are expressed as mean±S.E.M. (n=3). Statistical significance was determined by non-parametric Kruskal-Wallis test—Uncorrected Dunn's multiple comparisons test. *, p<0.05. **, p<0.01.

FIG. 5. AKBA alone and AKBA combinations—Comparison analysis of CB1R positive allosteric activity. A. Bar representation of CB1R activity (Fold change) of AKBA alone vs AKBA-THC combo treatments. B. Bar representation of CB1R activity (Fold change) of AKBA alone vs AKBA-THCA combo treatments. C. Bar representation of CB1R activity (Fold change) of AKBA alone vs AKBA-CBD combo treatments. Results are expressed as mean±S.E.M. (n=3). Statistical significance versus DLPA alone treatment was determined by ordinary one-way ANOVA—Fisher's LSD multiple comparisons test. *, p<0.05. **, p<0.01. ***, p<0.001. ****, p<0.0001.

### DETAILED DESCRIPTION OF THE INVENTION

The invention herein provides novel oral formulations comprising a combination of one or more cannabinoids with the natural health product Boswellia extract. The invention has a variety of advantages, including a surprising and synergistic effect for the treatment of human diseases, conditions and disorders. In particular, the oral formulation combinations provided herein demonstrate surprising and unexpected synergy for the treatment of a disease, condition or disorder selected from among pain, inflammation, anxiety, depression, sleep disorders, insomnia, lack of energy, lack of alertness, weight gain, obesity, diabetes, Metabolic Syndrome, acute and anticipatory nausea, suppressed appetite, epilepsy, spasticity, schizophrenia, bi-polar disorder, cancer and neoplasia, chronic pain, osteoarthritic pain, bacterial and/or fungal infections, fibromyalgia, and other disease, conditions and disorders disclosed herein.

As described herein, the invention is based on synergies identified by bioinformatics (overlapping sets of expressed genes induced by the combinations demonstrating mutually re-enforcing effects on specific biochemical pathways), and by testing the combinations in biological settings, including in vitro cell-based assays. Product embodiments exemplifying the invention are also provided. The invention has an objective of increasing safety, confidence, and enhanced treatment of the noted diseases and disorders with the claimed oral combinations and particular unit dosage forms.

The claimed Boswellia extract and cannabinoid compositions and their surprising synergy of effect has not been recognized by previous work, notably the disclosures published as WIPO publications WO2017007833 to George and WO2017066474 to Reynolds, and U.S. Pat. No. 9,861,611 to Bromely, U.S. Pat. No. 9,532,593 to Turner, U.S. Pat. No. 9,241,911 to Miller and U.S. Pat. No. 8,741,319 to Crain.

### DEFINITIONS

As used herein:

“Biomarker” means a measurable substance in the serum or tissue of an organism whose presence or level is indicative of a disease or condition. Biomarker presence or level will change (either increase or decrease) depending on the specific biomarker, and on the progress of the disease and the patient response to therapy.

“Cannabinoid” means any phytocannabinoid compound which 1) specifically binds to the human CB₁ receptor and/or CB₂ receptor under physiological conditions and which is 2) naturally synthesized by a plant (e.g. typically of the Cannabis species) or is a decarboxylated derivative thereof or is a liver metabolite thereof. Cannabinoids produced by Cannabis during its cultivation and growth include tetrahydrocannabinolic acid (THCA), cannabinolic acid (CBNA), cannabidiolic acid (CBDA) and cannabigerolic acid (CBGA). As used herein, cannabinoid also includes the corresponding decarboxylated moieties, tetrahydrocannabinol (THC), cannabinol (CBN), cannabidiol (CBD) and cannabigerol (CBG), (each of which may be derived from its parent compound by mild heating typically above 105° C.), and the corresponding liver metabolites that result upon oral consumption by humans of these compounds, such as but not limited to 11-OH-THC. Cannabinoids also include cannabichromene (CBC) and tetrahydrocannabivarin (THCV). Cannabinoids may be synthesized by chemical or biological methods. Phytocannabinoids may be distinguished from endocannabinoids which are chemically distinct, are synthesized in mammalian cells from lipids and other macromolecule precursors which are not phytocannabinoids, and are endogenous ligands of the CB₁ and/or CB₂ receptors.

“Cannabis” as used herein includes all members of the plant genus Cannabis, including without limitation C. sativa, C. indica, C. ruderalis, and hybrids thereof.

“Defined dose” means the dose of one or more active ingredients (typically cannabinoids) that has been selected during the production process and is signified to a consumer by a signifier associated with the oral formulation or Unit Dosage Form (UDF) of the invention.

“Natural Health Product” or “NHP” means a product which can be manufactured using sources from plants, algae, fungi or lichens, or other living matter. In some cases, an NHP may be dried plant matter, an extract, or a modification or a chemical derivative of a product found in the naturally occurring organism. NHPs are also known as dietary supplements or nutritional supplements in some contexts. They are typically regulated as foods and may be distinguished from drugs or pharmaceuticals which due to their powerful physiological effects and potential toxicities are more stringently regulated.

“Opaque” means tending to block transmission of visible light and/or UV-light, unless the context specifically indicates otherwise. “Substantially opaque” means substantially blocking including greater than or equal to than about 25%, 30%, 35%, 40%, 45%, 50%, 55%, 60%, 65%, 70%, 75%, 80%, 90%, 95%, 96%, 97%, 98%, 99%, and 100% blocking.

“Oral formulation” means a formulation which is conveniently administered orally to a human subject.

“Pharmacodynamic” parameters (PD) means dose-response relationships, that is, the relationships between a substances' plasma concentration and its effect.

“Pharmacokinetic” (PK) parameters are usually used to describe the rate of absorption of a substance into a biological system. Graphing a substance's serum concentration versus time reveals of the drug's basic PK properties: the maximum concentration the drug attains (Cₘₐₓ), the time at which this maximum concentration occurs (tₘₐₓ), and the area under the concentration-versus-time curve (AUC) which estimates total systemic exposure.

“Therapeutically effective amount” means an amount sufficient to elicit an objective or subjective therapeutic response to a disease or a condition in an individual. In the case of a unit dosage form, a therapeutically effective amount means one or more doses of the specific unit dosage form. For chronic conditions it may mean one or more doses per day or per week. In some embodiments a therapeutically effective amount will mean consumption of multiple UDF doses per day.

“Unit dosage form” or “UDF” means a physically fixed unit dose of a formulation which is conveniently consumed by a consumer in unit form (e.g. requires no measuring or adjusting of dosage before consumption). A consumer may consume one or more UDFs at a time.

### Key Ingredients of the Unit Dosage Form

The compositions and methods of the invention feature a combination of Boswellia extract and one or more cannabinoids. These components may be sourced as follows.

Boswellia serrata is a moderate-to-large branching tree that grows in high, dry habitats in India, Northern Africa, and the Middle East. Its gummy resin is tapped from the tree by means of incisions in its bark that allow the thick, oily substance to ooze out for collection and drying (Siddiqui, 2011). The resin, which is thick and oily, is further extracted with organic, aqueous or mixed organic-aqueous solvents to generate a Boswellia extract that can be used for its anti-inflammatory, pain relieving, arthritis-fighting, and anticancer effects (Pandey et al 2005). Reference to Boswellia extract in this specification refers to an extract generated from Boswellia resin, or compounds found in an extract of the resin of Boswellia serrata or any other member of the Boswellia species, such as Boswellia carteri, Boswellia frereana, and Boswellia sacra. These compounds may be extracted from the resin or they may be synthesized by chemical or biochemical means. The compounds found in Boswellia extract include 3-O-acetyl-11-ketobeta-boswellic acid (AKBA), also known as 3-Acetyl-11-keto-β-boswellic acid (AKBA) (Molecular Weight: 512.72; CAS Number: 67416-61-9). AKBA may also refer to purified or semi-purified forms of AKBA which are 11-keto-β-Boswellic acid acetate (AKBA) and 3α-Acetoxy-11-oxo-12-ursen-24-oic acid (AKBA). Additional compounds found in Boswellia extract include α-Boswellic acid ((3α,4β)-3-Hydroxyolean-12-en-23-oic acid, 3≢-Hydroxyolean-12-en-24-oic acid; Empirical Formula (Hill Notation): C₃₀H₄₈O₃; Molecular Weight: 456.70; CAS Number: 471-66-9), β-Boswellic acid ((3α,4β)-3-Hydroxyurs-12-en-23-oic acid, 3α-Hydroxyurs-12-en-24-oic acid; Empirical Formula (Hill Notation): C₃₀H₄₈O₃; Molecular Weight: 456.70; CAS Number: 631-69-6), Boswellic acid (typically a mixture of α and β forms), 3-O-Acetyl-α-boswellic acid (α-Boswellic acid acetate, (3α,4β)-3-(Acetyloxy)olean-12-en-23-oic acid, 3α-(Acetyloxy)olean-12-en-24-oic acid; Empirical Formula (Hill Notation): C₃₂H₅₀O₄; Molecular Weight: 498.74; CAS Number: 89913-60-0), 3-O-Acetyl-β-boswellic acid (β-Boswellic acid acetate, (3α,4β)-3-(Acetyloxy)urs-12-en-23-oic acid, 3α-Hydroxyurs-12-en-24-oic acid acetate; Empirical Formula (Hill Notation): C₃₂H₅₀O₄; Molecular Weight: 498.74; CAS Number: 5968-70-7), and 11-Keto-β-boswellic acid (11-Oxo-β-boswellic acid, 3α-Hydroxy-11-oxours-12-en-24-oic acid, KBA; Empirical Formula (Hill Notation): C₃₀H₄₆O₄; Molecular Weight: 470.68; CAS Number: 17019-92-0). Reference to “Boswellia extract” herein may refer to any of the compounds individually, or a mixture of such compounds (as found in an extract of Boswellia resin), unless the context specifically indicates otherwise.

Boswellia extract can be administered at dosages of 100, 200, 300, 400, 500, 600, 700, 800, 900, and 1,000 mg (QD)×1 daily therapeutic regimen. A Boswellia extract of botanical origin typically contains 15-85% by weight of the Boswellic acid compounds listed above (depending on process). The oleo-gum resin, obtained by incision of the bark, is composed by essential oil (5-9%), mucopolysaccarides (21-22%), and pure resin (65-85%), containing tetracyclic and pentacyclic triterpene acids, of which boswellic acids (BAs) are the most important bioactive molecules. Preferably the starting form is standardized to 60% by weight of Boswellic acid compounds. AKBA itself may comprise most commonly 10-60% by weight, and is sometimes 40% by weight. The Health Canada NHP monograph for joint health sets the maximum daily dose of Boswellia serrata extract at 1,000 mg standardized to 40% Boswellic acid. This dosage administered in 3 divided doses was shown to be beneficial for the relief of joint pain associated with osteoarthritis and is in compliance with the recommended Health Canada requirements outlined in the Natural Health Products Ingredient Database (NHPID).

The unit dosage form of the invention further comprises a defined dose of one or more cannabinoids selected from among the group consisting of
0.1-750 mg tetrahydrocannabinolic acid (THCA),
0.1-100 mg tetrahydrocannabinol (THC),
0.1-750 mg cannabidiolic acid (CBDA),
0.1-750 mg cannabidiol (CBD), 0.1-750 mg cannabichromene (CBC), and
0.1-750 mg cannabigerol (CBG).

The cannabinoid(s) may be prepared as an extract of a cultivated Cannabis plant crop (as described further below), by a biosynthetic process or they may be synthetically prepared in a chemical process (as for example in patent applications WO2014134281, WO2015068052, WO2016030828 and others in the name of Full Spectrum Laboratories Limited (Dublin IE)). When prepared as an extract, the composition may also comprise terpenes and other organic molecules co-extracted in the process.

As will be described further below, the unit dosage form may also comprise diverse additional features which may include an anti-oxidant, other pharmaceutically acceptable additives, a carrier oil, an outer shell that is substantially opaque to one or both of ultraviolet and visible light, an enteric-coating, and/or a signifier which signifies the cannabinoid dosage of the unit dosage form, such as a signifier generated by embossing, or by colour, pattern or shape feature, which signifier may be adapted to be directly interpreted by a consumer and/or is a machine-readable code.

The inventors have identified the advantages of the proposed novel combinations based on a variety of technical assessments which demonstrate surprising and synergistic effects of the oral combinations on metabolic pathways relevant to treatment of specific human diseases and disorders. These assessments, described below, include 1) Identifying sets of expressed genes that are mutually activated by the combination (thus permitting stronger effects to be achieved using lower doses, as further described below); and 2) Identifying mutually interacting effects on specific biochemical pathways through chemical-protein interactions, herein identified as agonistic, allosteric and/or antagonistic effects.

### Utility of the Invention

Based on the technical assessments disclosed herein, the oral formulation of the invention is provided as a therapeutic and natural health product agent for the treatment or amelioration in humans and other animals of a range of diseases, conditions and disorders. These diseases, conditions and disorders are selected from among the group consisting of pain, inflammation, anxiety, depression, sleep disorders, insomnia, lack of energy, lack of alertness, weight gain, obesity, diabetes, Metabolic Syndrome, acute and anticipatory nausea, suppressed appetite, epilepsy, spasticity, schizophrenia, bi-polar disorder, cancer and neoplasia, chronic pain, osteoarthritic pain, fibromyalgia, all the foregoing in acute and/or chronic presentation, and can be further used to induce appetite suppression and/or act as an anti-proliferative agent (for the treatment of neoplasia or cancer).

Additionally, the oral formulation can be used to treat or ameliorate Inflammatory Bowel Disease (IBD), Crohn's Disease (CD), arthritis (including osteoarthritis and rheumatoid arthritis, and other forms), cardiovascular Inflammation, ischemic heart disease, neuroprotection, and for use in treating muscle aches, persistent arthritis related pain, nociceptive and neuropathic pain, such as post-herpetic neuralgia, trigeminal neuralgia, diabetic neuralgia, and postoperative or posttraumatic pain, as well as endogenous depression, ADHD and symptoms of Parkinson's disease, Huntington's disease, Multiple Sclerosis, drug and alcohol dependence, asthma, allergic hypersensitivity, uveitis, eosinophilia, peritonitis, gastritis, exanthem, periodontitis, thrombocytopenia, pain agnosia, toxic shock syndrome, treatment of infectious diseases (including malaria, influenza and human immunodeficiency virus), anemia, lung diseases, neurological diseases, liver diseases, metabolic diseases, autoimmune diseases, cardiovascular diseases, hypoglycemia, wound healing, anti-microbial activities, psoriasis, ulcerative proctitis, ulcerative colitis, alveolar osteitis (dry socket), proliferative vitreoretinopathy (PVR), loss of appetite, abdominal cramps, diarrhea control, allodynia, medication-rebound headache, b-amyloid-induced neuroinflammation, bacterial and/or fungal infections, reperfusion injury, autoimmune encephalomyelitis, acute lung injury, Alzheimer's disease, CNS inflammation, major depressive disorder, treatment resistant depression, anxiety disorders, post-traumatic stress disorder (PTSD), treatment of nightmares, PTSD-associated insomnia, other PTSD symptoms, toxic encephalopathy, cerebrovascular disease, hypertension, hyperglycemia, coronary artery disease, cardiomyopathy including hypertrophic and dilated cardiomyopathy, spinal cord injury, dementia, collagen disease, vasculitis, leukopenia and fatty liver disease, peripheral neuropathies (such as diabetic neuropathy, chemotherapy-induced peripheral neuropathy, carpal tunnel syndrome, sciatic pain, low-back pain, failed back surgery syndrome, dental pains, neuropathic pain in stroke, chronic pelvic pain, post-herpetic neuralgia, and vaginal pains), endometriosis-associated pain, neurohypophyseal diabetes, amnestic disorder, hypoglycemia, neonatal jaundice, diabetes insipidus, chronic kidney disease, ovarian hyperstimulation syndrome, Kuhnt Junius degeneration, capillary hemangioma, brain edema, cystinuria, portal hypertension, Coats' disease, and to provide immunosuppression.

The overlapping biochemical pathways of the combination of the invention also indicate that treatment is indicated for any disease, disorder or condition identified by abnormal levels (either excess or deficiency) of serum biochemical markers such as IL-6, IL-8, MCP-1, COX-2, IκBα, IL-1α, IL-1β, MKP-1, TNFα and C-reactive protein. Similarly, response to therapy can be observed by the impact on these serum biomarkers after administration of an oral formulation of the invention to a human subject.

### Production of Unit Dosage Forms of the Invention

In some embodiments, the capsules of the invention are prepared from a Boswellia extract, or from a more purified form of AKBA or Boswellic acid (CAS Number (α/β): 471-66-9/631-69-6). Boswellic acid may be used in various forms such as α-Boswellic acid, β-Boswellic acid, or a mixture of α and β forms. Boswellic acid may be commercially sourced in powdered or liquid form. Suitable sources of powdered Boswellic acid for embodiments of this invention include, but are not limited to, Millipore Sigma (α-Boswellic acid SKU: 06813, β-Boswellic acid SKU: 80342, Boswellic acid—mixture of α and β SKU: 63850), Banyan Botanicals (Boswellia Powder), Bonanza (100% Pure Herb Boswellia Seratta Extract Powder), Mountain Maus Remedies (Frankincense Tears Powder) and Starwest Botanicals (Frankincense Tears & Powder). Suitable sources of liquid Boswellic acid include, but are not limited to, Now Health Group Inc. (Frankincense Oil—100% Pure Boswellia carterii, Upaya Naturals (Frankincense—Boswellia Essential Oil, Product Code: DOT-016), Ice N Fire (Frankincense Ice E Oil Pure Essential Oil), US Organic (100% Pure Organic—Frankincense Essential Oil), Vitruvi (Frankincense Essential Oil), and Now Foods (Essential Oil—Frankincense, Product Code: NOW-07542).

Powders
https://www.sigmaaldrich.com/catalog/search?term=boswellic+acid&interface=All&N=0&mode=partialmax&lang=en&region=CA&focus=product
Banyan Botanicals, https://www.banyanbotanicals.com/boswellia-powder/
Bonanza, https://www.bonanza.com/listings/100-Pure-Herb-Boswellia-Seratta-Extract-Powder-Raw-Joint-Health-100g-Grade-A/457900693?currency=CAD&goog_pla=1&gpid=293946777986&keyword=&goog_pla=1&pos=1o1&ad_type=pla&agid=56644786559&cid=1485578864&gclid=Cj0KCQjwquTbBRCSARIsADzW88yXFoFEVllgskjrqPO8x8sGPp8Uzf_xyv4n4QBXgeti99Xeg-MGpmsaAsN5EALw_wcB
Mountain Maus Remedies, http://www.mountainmausremedies.com/frankincense-tears-powder/
Starwest Botanicals, https://www.starwest-botanicals.com/category/frankincense/

Liquids:
Now Health Group, Inc., https://www.nowfoods.ca/essential-oils/frankincense-oil-ca
Upaya Naturals,
https://www.upayanaturals.com/Frankincense_Boswellia_Essential_Oil_Essential_p/dot-016.htm?gclid=Cj0KCQjwquTbBRCSARIsADzW88w2BMAqnW6zwDRlQI8lihIBFXmceXh3RO_UeASLRefir900JmyNICIaAkpXEALw_wcB
Ice N Fire, https://www.icenfire.ca/products/frankincense-ice-e-oil?variant=95268372486&utm_medium=cpc&utm_source=google&utm_campaign=Google%20Shopping&gclid=Cj0KCQjwquTbBRCSARIsADzW88yo_AfPfdT46PdjsE8fvUoSQzstbdwsQ2PI8xTmYACfCcUAAz3-AC4aAn73EALw_wcB
US Organic, https://www.us-organic.com/products/100-pure-certified-usda-organic-frankincense-boswellia-carteri-essential-oil-10ml
Vitruvi, https://vitruvi.ca/products/frankincense-essential-oil?variant=2571641421860&utm_medium=cpc&utm_source=google&utm_campaign=Google%20Shopping&gclid=Cj0KCQjwquTbBRCSARIsADzW88w1o2RaRaABfBC1eTXZFeunzBp4SlD5bfbbZg8bFXbptcSP0eb2AgsaAmXjEALw_wcB
Now Foods, https://ca.iherb.com/pr/Now-Foods-Essential-Oils-Frankincense-1-fl-oz-30-ml/2480?gclid=Cj0KCQjwquTbBRCSARIsADzW88y-KOiJrJ1Wk_zRezcIt6EDeL9ByRNWh3w0-yYiyFpq8FKNi0dIbKoaAifUEALw_wcB&gclsrc=aw.ds

As described above, the cannabinoids in the oral formulation of the present invention may be provided as dried plant matter, as extracts of plant matter, or as generated by chemical or biosynthetic synthesis. A valuable parameter for usefulness is on whether the format is flowable. The cannabinoid format is preferably efficient for mixing for loading into capsules, forming tablets, and the like. Stickiness of powders or high-viscosity of liquids can be a deterrent to efficient preparation. The invention therefore takes advantage cannabinoid formats that are sufficiently flowable for use in manufacturing the formulations described herein. Flowability of dried plant material may be enhanced by appropriate grinding and by addition of excipients, including but not limited to those described herein. Flowability of oil extracts may be enhanced by diluents, gliders and the like. Oil extracts with hydrophobic components may be mixed with powders to provide a dry flowable powder which can easily be mixed with other formulation components. (e.g., US Pat App. Publications 20170232210 and 20160243177, incorporated herein by reference). Alternatively, oil extracts may be spray dried with flowable particles to create a flowable powder format. In one preferred embodiment, extracts may be used to produce crystallized pure cannabinoids. Crystallized CBD can be prepared by high-vacuum treatment of extracts, as exemplified at https://www.leafscience.com/2017/11/06/cbd-isolate-powder/ (viewed 22 Aug. 2018). Crystallized forms can be rendered into a suitable flowable powder by techniques common in the capsule/tablet industry.

Boswellia extract can be administered at dosages of 100, 200, 300, 400, 500, 600, 700, 800, 900, and 1,000 mg (QD)×1 daily or (BID)×2 daily or (TID)×3 daily therapeutic regimens. The Health Canada NHP monograph for joint health sets the maximum daily dose of Boswellia serrata extract at 1,000 mg standardized to 40% boswellic acid. This dosage administered in 3 divided doses was shown to be beneficial for the relief of joint pain associated with osteoarthritis and is in compliance with the recommended Health Canada requirements outlined in the Natural Health Products Ingredient Database (NHPID).

In some embodiments, the dosage of Boswellia extract provided in the dosage forms of the present disclosure is about 100 mg to 1000 mg, or about 250 mg, about 450 mg, or about 1000 mg.

The oral combinations of the invention further comprise one or more cannabinoids selected from among the group consisting of:
0.1-750 mg tetrahydrocannabinolic acid (THCA),
0.1-100 mg tetrahydrocannabinol (THC),
0.1-750 mg cannabidiolic acid (CBDA),
0.1-750 mg cannabidiol (CBD), 0.1-750 mg cannabichromene (CBC), and
0.1-750 mg cannabigerol (CBG);

In some embodiments, the oral combinations may comprise a defined dose selected from the following ranges (which may be referred to as “low dose”): about 0 mg, 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg THC, about 0 mg, 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg THCA, about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or about any 1 mg interval between 0 mg and 100 mg CBD, and/or about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or about any 1 mg interval between 0 mg and 100 mg CBDA, or 10 mg, 20 mg, 30mg, 40 mg, 50 mg, 60 mg, 70 mg, 80 mg, 90 mg, 100 mg, or about any 1 mg interval between 0 mg and 100 mg CBG, and/or about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or about any 1 mg interval between 0 mg and 100 mg CBC. In some embodiments, the oral combinations comprise about 0 mg, 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or about any 1 mg interval between 0 mg and 100 mg of one of the foregoing compounds. In some embodiments, the oral combinations of the present invention have defined dosages for more than one of the foregoing compounds. For example, in some embodiments, the oral combinations comprise from about 0 mg-1 mg, or any 0.1 mg interval therebetween THC, about 0 mg, about 9 mg, about 90 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg, about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg CBD, and/or about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg CBDA. In some embodiments, the oral combinations comprise from about 0 mg, about 9 mg, about 90 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg THC, 0 mg-1 mg THCA, or any 0.1 mg interval therebetween, about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg CBD, and about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg CBDA. In some embodiments, the compositions are substantially free of THC-type cannabinoid compounds. For example, in some embodiments the oral combinations comprise from about 0 mg-1 mg, or any 0.1 mg interval therebetween THC, 0 mg-1 mg THCA, or any 0.1 mg interval therebetween, about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg CBD, and about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg CBDA.

In some embodiments, the oral combinations may comprise a defined dose selected from the following ranges (which may be referred to as “high dose”): about 10 mg, 15, 20, 25, 30, 40, 50, 60, 70, 80, 90, or 100 mg, 200 mg, 300 mg, 400 mg, 500 mg, 600 mg, 700 mg, 750 mg, or any about any 10 mg interval between 0 mg and 750 mg THCA, about 0 mg, 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg THC, about 10 mg, 15, 20, 25, 30, 40, 50, 60, 70, 80, 90, or 100 mg, 200 mg, 300 mg, 400 mg, 500 mg, 600 mg, 700 mg, 750 mg, or any about any 10 mg interval between 0 mg and 750 mg CBD, and/or about 10 mg, 15, 20, 25, 30, 40, 50, 60, 70, 80, 90, or 100 mg, 200 mg, 300 mg, 400 mg, 500 mg, 600 mg, 700 mg, 750 mg, or any about any 10 mg interval between 0 mg and 750 mg CBDA, or about 10 mg, 15, 20, 25, 30, 40, 50, 60, 70, 80, 90, or 100 mg, 200 mg, 300 mg, 400 mg, 500 mg, 600 mg, 700 mg, 750 mg, or any about any 10 mg interval between 0 mg and 750 mg CBG, and/or about 10 mg, 15, 20, 25, 30, 40, 50, 60, 70, 80, 90, or 100 mg, 200 mg, 300 mg, 400 mg, 500 mg, 600 mg, 700 mg, 750 mg, or any about any 10 mg interval between 0 mg and 750 mg CBC. In some “high dose” embodiments, the oral combinations comprise about 10 mg, 15, 20, 25, 30, 40, 50, 60, 70, 80, 90, or 100 mg, 200 mg, 300 mg, 400 mg, 500 mg, 600 mg, 700 mg, 750 mg, or any about 10 mg interval between 0 mg and 750 mg of one of the foregoing compounds. In some “high dose” embodiments, the oral combinations of the present invention have defined dosages of more than one of the cannabinoids. In some high dose embodiments, the compositions are substantially free of THC-type cannabinoid compounds. For example, in some embodiments the oral combinations comprise from about 0 mg-1 mg, or any 0.1 mg interval therebetween THC, 0 mg-1 mg THCA, or any 0.1 mg interval therebetween, plus CBD in the amount of about 0 mg, about 25 mg, about 75 mg, about 10 mg, 20, 30, 40, 50, 60, 70, 80, 90, or 100 mg, 200 mg, 300 mg, 400 mg, 500 mg, 600 mg, 700 mg, 750 mg, or any about 10 mg interval between 0 mg and 750 mg, and/or CBDA and/or CBC in the amount of about 0 mg, about 25 mg, about 75 mg, about 10 mg, 20, 30, 40, 50, 60, 70, 80, 90, or 100 mg, 200 mg, 300 mg, 400 mg, 500 mg, 600 mg, 700 mg, 750 mg.

In some embodiments, the oral combinations described herein comprise an “effective” amount of one or more of the cannabinoid ingredients described herein. The term “effective amount” refers to an amount of the one or more cannabinoid ingredients sufficient to induce a response in an individual user, either subjectively or objectively determined. An effective amount also means an amount of the one or more cannabinoid ingredients that is needed to provide a desired level of cannabinoid(s) in the bloodstream of an individual user to provide an anticipated physiological response. An effective amount of a cannabinoid ingredient can be administered in one administration, or through multiple administrations of an amount that totals an effective amount, preferably within a 24-hour period. It is understood that the effective amount can be the result of empirical and/or individualized (case-by-case) determination on the part of the individual user. For example, a therapeutically effective amount of said one or more cannabinoid ingredients may be in the range of about 1 mg to 2,000 mg, or any 1 mg or 10 mg interval therebetween total cannabinoids per day.

In some low dose embodiments, an effective amount of said one or more cannabinoid ingredients may be in the range of about 1 mg-5 mg, or any 1 mg or 0.1 mg interval therebetween per day. For example, for an adult, about 1-2 mg, or 0.1 mg interval therebetween, per day total of THC may provide a very low end dose below the psychoactive threshold.

In some embodiments, an effective amount of THC may be in the range of about 5 mg-25 mg, or any 1 mg interval therebetween. For example, most vapers inhale about 10 to 30 mg of THC to establish a mild, temporary, psychoactive effect. In a high dose embodiment, the oral formulation may contain THC in an amount of 25 mg to 100 mg.

In some embodiments, a composition of the present invention may comprise THCA in an amount between 5-200 mg, THC in an amount less than 1.0 mg, and CBDA in an amount between 0.1-600 mg, and have a total mass of 100-750 mg.

In some embodiments, a composition of the present invention may comprise THCA in an amount less than 5.0 mg, THC in an amount between 5-30 mg, and CBD in an amount between 0.1-600 mg, and have a total mass of 100-750 mg.

In some embodiments, a composition of the present invention may comprise THCA in an amount less than 1.0 mg, THC in an amount less than 1.0 mg, and CBD in an amount between 5-600 mg, and have a total mass of 100-750 mg.

In some embodiments, a composition of the present invention may comprise THCA in an amount less than 1.0 mg, THC in an amount less than 1.0 mg, and CBG in an amount between 5-600 mg, and have a total mass of 100-750 mg.

In some embodiments, an effective amount of CBD or CBC for treating conditions or disorders disclosed elsewhere herein may be in the low dose range of about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg per day. Preferably, the low dose amount of CBD may be about 50 mg per day. For example, a recommended CBD or CBC dosage standard may be about 25 mg of CBD or CBC taken twice a day.

Alternatively, in some embodiments, an effective amount of CBD or CBC for treating conditions or disorders disclosed elsewhere herein may be in the high dose range of about 50-2000 mg/day or higher. Such effective amounts may be provided by ingestion of multiple oral dosage forms comprising CBD or CBC in the amount of, about 50 mg, about 75 mg, about 100 mg, 200 mg, 250 mg, 300 mg, 400 mg, 500 mg, 600 mg, 700 mg, 750 mg, or any about any 10 mg interval between 100 mg and 750 mg.

In some embodiments, an effective amount of THCA may be in the range of about 0 mg, about 9 mg, about 90 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg.

In some embodiments, only one cannabinoid will be present at a physiologically relevant level, in other embodiments two or more cannabinoids may be present at physiologically relevant levels. The second cannabinoid may be one previously cited or may be an alternative cannabinoid which may also be isolated from or extracted from Cannabis, or may be a simple chemical derivative thereof. Table 1 provides examples for preferred embodiments.

TABLE 1
Preferred cannabinoid doses (single or in combination) of the UDF.
Table 1A-50 mg capsule (low dose)

| | Pre- | | | | | | | | |
|---|---|---|---|---|---|---|---|---|---|
| Form | dominant | Active Ingredient (mg/cap) | | | | | | | |
| # | Cannabinoids(s) | THCA | THC | CBDA | CBD | CBG | CBC | THCV | Indication |
| 1 | THCA | 25 | 2 | | | | | | Pain, Acute and anticipatory nausea; |
| | | | | | | | | | Obesity, Metabolic Syndrome |
| 2 | THC | | 25 | | | | | | Pain, Appetite enhancement |
| 3 | CBDA | 1 | | 25 | 2 | | | | Acute and anticipatory nausea |
| 4 | CBD | | 1 | | 25 | | | | Anxiety, Sleep |
| 5 | THCA:CBDA | 25 | 2 | 25 | 2 | | | | Acute and anticipatory nausea; |
| | | | | | | | | | Obesity, Metabolic Syndrome |
| 6 | THCA:CBD | 25 | 2 | | 25 | | | | Pain; Anxiety; Sleep; Obesity, |
| | | | | | | | | | Metabolic Syndrome |
| 7 | THC:CBD | | 25 | | 25 | | | | Pain; Anxiety; Sleep |
| 8 | THC:CBD | | 25 | | 2 | | | | Energy |
| 9 | CBD:CBG:CBC | | 1 | | 25 | 25 | 25 | | Osteoarthritic Pain |
| 10 | THC:THCV | | 25 | | | | | 25 | Energy |

Table 1B-250 mg capsule (low dose; 10 mg THC maximum)

| | Pre- | | | | | | | | |
|---|---|---|---|---|---|---|---|---|---|
| Form | dominant | | | | | | | | |
| # | Cannabinoids(s) | THCA | THC | CBDA | CBD | CBG | CBC | THCV | Indication |
| 11 | THCA | 9 | 1 | | | | | | Pain, Acute and anticipatory nausea; |
| | | | | | | | | | Obesity, Metabolic Syndrome |
| 12 | THC | | 10 | | | | | | Pain, Appetite enhancement |
| 13 | THCA:CBDA | 9 | 1 | 9 | 1 | | | | Acute and anticipatory nausea; |
| | | | | | | | | | Obesity, Metabolic Syndrome |
| 14 | THCA:CBD | 9 | 1 | | 10 | | | | Pain; Anxiety; Sleep |
| 15 | THC:CBD | | 10 | | 10 | | | | Pain; Anxiety; Sleep |
| 16 | THC:CBD | | 10 | | 1 | | | | Energy |
| 17 | THC:THCV | | 10 | | | | | 10 | Energy |

Table 1C-1000 mg capsule (high dose)

| | Pre- | | | | | | | | |
|---|---|---|---|---|---|---|---|---|---|
| Form | dominant | | | | | | | | |
| # | cannabinoids(s) | THCA | THC | CBDA | CBD | CBG | CBC | THCV | Indication/fuction |
| 19 | THCA | 600 | | | | | | | Pain; Acute and anticipatory nausea; |
| | | | | | | | | | Obesity, Metabolic Syndrome |
| 20 | THCA:THC | 600 | 60 | | | | | | Pain; Obesity, Metabolic |
| | | | | | | | | | Syndrome |
| 21 | THC | | 100 | | | | | | Pain; |
| 22 | CBDA | | | 600 | | | | | Acute and anticipatory nausea |
| 23 | CBDA:CBD | 25 | | 600 | 60 | | | | Acute and anticipatory nausea |
| 24 | CBD | | | | 100 | | | | Anti-epileptic |
| 25 | CBD | | 4 | | 100 | | | | Anti-epileptic |
| 26 | CBD | | | | 600 | | | | Anti-epileptic |
| 27 | CBD | | 25 | | 600 | | | | Chronic Pain; Inflammation; |
| | | | | | | | | | Schizophrenia; Cancer Anti- |
| | | | | | | | | | proliferative |
| 28 | CBG | | | | | 600 | | | Cancer Anti-proliferative; |
| | | | | | | | | | Antimicrobial; bone stimulant |
| 29 | THCA:CBDA | 300 | | 300 | | | | | Acute and anticipatory nausea; |
| | | | | | | | | | Obesity, Metabolic Syndrome |
| 30 | THCA:CBDA | 300 | 30 | 300 | 30 | | | | Acute and anticipatory nausea, |
| | | | | | | | | | Obesity, Metabolic Syndrome |
| 31 | THCA:CBD | 300 | | | 300 | | | | Pain; |
| 32 | THCA:CBD | 300 | 30 | | 300 | | | | Pain; |
| 33 | THC:CBD | | 100 | | 100 | | | | Pain; Spasticity; |
| 34 | THC:CBD | | 100 | | 30 | | | | Pain; |
| 35 | THC:CBG | | 300 | | | 300 | | | Pain; Cancer Anti-proliferative |
| 36 | THC:CBC | | 300 | | | | 300 | | Pain; Anti-Inflammatory |
| 37 | CBD:CBG | | | | 300 | 300 | | | Pain; Cancer Anti-proliferative |
| 38 | CBD:CBC | | | | 300 | | 300 | | Pain; Anti-Inflammatory |
| 39 | CBD:CBG:CBC | | | | 300 | 300 | 300 | | Osteoarthritic Pain; Anti- |
| | | | | | | | | | proliferative |
| 40 | CBD:CBG:CBC | | 10 | | 250 | 250 | 250 | | Osteoarthritic Pain; Anti- |
| | | | | | | | | | proliferative, bacterial and/or |
| | | | | | | | | | fungal infections |
| 41 | THC:THCV | | 100 | | | | | 500 | Pain; |
| 42 | CBD:THCV | | | | 300 | | | 300 | Appetite suppression; |
| 43 | CBD:THCV | | | | 100 | | | 100 | Anti-epileptic |

The precise amount of cannabinoid required for a therapeutically effective dose in an individual will depend upon numerous factors, e.g. type of cannabinoid(s) and type of natural health product, and the synergistic effect of the combination. This disclosure provides UDFs suitable to obtain a therapeutically effective dose which can be determined subjectively by the user or objectively by methods known to those skilled in the art.

An achievement of the invention is that by using the UDF of the invention, users and medical advisors for the first time have knowledge of and certainty with the exact doses of cannabinoid they are employing with Boswellia extract. This is preferably achieved with a signifier identifying dosage of one or more components, as detailed further below.

### Source and Quality of Cannabinoid

The cannabinoid(s) may be prepared by a variety of methods. It may be provided in the original plant form, preferably dried and cured into a flowable powder suitable for encapsulating. An alternative preferred method is by extraction from a cultivated Cannabis crop. Organic extraction is a preferred method, although aqueous extraction, typically employed to prepare hashish, is also possible. Organic extraction can be performed with a wide variety of organic solvents or super-critical carbon dioxide, and at a variety of temperatures and under a variety of conditions. (Fairbairn and Liebmann (1973) J. Pharm. Pharmac. 25:150-155; Romano and Hazekamp (2013) Cannabinoids. 1(1)-1-11; Rovetto and Aieta (2017). J. Supercritical Fluids. 129: 16-27.), each of which references is incorporated herein by reference in its entirety. The resulting organic solvent-based extract can be, at room temperature, a liquid oil, or solid form wax, budder or shatter depending on the conditions employed (which significantly impact the other plant alkyloids and polymers extracted by the process). Historically, less than 50% of cannabinoids were extracted from dried plant material. (Fairbairn and Liebmann (1973)). Modern techniques may extract over 90%.

The unpredictability of cultivated Cannabis is another challenge that must be overcome. As is well known, the most common varieties C. sativa, C. indica and C. ruderalis, have distinct (but overlapping) ranges of cannabinoids. Varieties and strains which are crossed or hybridized generate further different cannabinoid ratios. And, the cannabinoid ratios and overall amounts within a single variety are strongly influenced by the conditions of cultivation, especially light cycle, temperature, soil condition, nutrient availability, timing of harvest and pathogen exposure. The result is that a cultivated Cannabis can have, by dry weight, anywhere from 0% up to greater than 30% of selected cannabinoids, and the ratios between individual cannabinoids can be highly diverse.

Preferred Cannabis sp. cultivars for use preparing cannabinoid extracts include Time Warp A3 (Hybrid, THC); Island Honey; Blue God; OGMB; Critical Call Mist; Sensi Little Twin; Nukem; Sensi Big Twin; Blueberry Kush; Afghani Kush; Crystal Kush; Big Bud XXL; Ocean Pearl; Critical Kush; K8; MK Tonic; Saltspring King; Purple X Chemo; Hash Plant (Indica, THC); White Rhino (Indica, THC); Master Kush (Indica, THC); Headband (Hybrid, THC); AK47 (Hybrid, THC); Armageddon (Hybrid, THC); Critical Kali Mist (Sativa, THC); Blue Cheese (Indica, THC); CBD Shark (Indica, THC); Sour Diesel (Sativa, THC); Durban Poison (Sativa, THC); Blue Cheese (Indica, THC); Acapulco Gold; Afghani; African; Cambodian red; Columbian; Hawaiian; Jamaican gold; Mexican red; Panama red; Thai stick; Amnesia; AK-47; Amnesia Haze; Blueberry; Blue Dream; Bubba Kush; Bubblegum; Critical Mass; Durban Poison; Gorilla Glue; Haze; Hindu Kush; Jack Herer; Maui Waui; Northern Lights; OG Kush; Purple Haze; and Skunk. Preferred for cultivation in Canada are: Altair, Angie, CS, Carmagnola, Carmen, Deni, ESTA-1, FINOLA, Fasamo, Fedrina 74, Felina 34, Fibranova, Fibriko, Fibrimon 24, Fibrimon 56, Georgina, GranMa, Grandi, Judy, Katani, Kompolti, Kompolti Hibrid TC, Kompolti Sargaszaru, Laura Secord, Lovrin 110, Martha, Petera, Picolo, Quida, UC-RGM, Uniko B, Victoria, and Yvonne.

Preferred Cannabis sp. cultivars for use in preparing CBD extracts that contain little or no THC or THCA include: Charlotte's Web, Island Mist (Sativa, CBD), ACDC (Hybrid, CDB), Harle TSU (Hybrid, CBD), and cultivars approved in Canada including CFX-1, CFX-2, CRS-1, Canda, Crag, Joey, USO 14, USO 31, X-59 (Hemp Nut), Delores, Silesia, Alyssa, Zolotonosha 11, Anka, Jutta, CanMa, and Ferimon.

Another critical aspect of the cannabinoid preparation is that during the cultivation phase, Cannabis naturally synthesizes tetrahydrocannabinolic acid (THCA) and cannabidiolic acid (CBDA). These compounds convert respectively to THC (the primary psychoactive cannabinoid), and CBD (the non-psychoactive analgesic and anti-inflammatory cannabinoid) via decarboxylation. Decarboxylation may be induced by heating over 105° C. and/or by exposure to ultraviolet (UV) light. Significantly, gastric acids do not decarboxylate THCA or CBDA. (See Wang et al. (2016) Decarboxylation Study of Acidic Cannabinoids: A Novel Approach Using Ultra-High-Performance Supercritical Fluid Chromatography/Photodiode Array-Mass Spectrometry. Cannabis Cannabinoid Res.; 1(1): 262-271.) Therefore, a critical aspect of extracted cannabinoids is quality control on whether the harvested crop has been subjected to decarboxylating conditions that would decarboxylate THCA and CBDA to THC and CBD, respectively.

Additionally, minor cannabinoids may be present in certain strains at therapeutically useful levels. Cannabichromene (CBC) is a non-psychoactive cannabinoid widely considered to interact with the endocannabinoid system (ECS) through stimulation of the body's naturally occurring endocannabinoids, anandamide and 2-AG, and is a known agonist to TRPV1 and TRPA1 receptors (A. A. Izzo et al.: Br. J. Pharmacol. 166, 1444 (2012)). Furthermore, CBC is thought to be a selective CB2 receptor agonist which may have therapeutic implications for the treatment of pain and inflammatory conditions through CB2-mediated regulatory pathways (M. Udoh et al.: Br. J. Pharmacol. (2019). Furthermore, CBC co-administered with THC produced an enhanced anti-inflammatory effect, suggesting a potential pharmacokinetic interaction between the two molecules (G. T. DeLong et al.: Drug Alcohol Depend. 112, 126 (2010). CBC has been identified as a molecule of interest for various therapeutic applications including pain, inflammation, digestive and gastrointestinal disorders. Additionally, it is known to have antibacterial and antifungal effects, and could potentially contribute to the regeneration of brain cells, which possibly has implications in the treatments of multiple sclerosis, fibromyalgia, dementia, Alzheimer's and other neurodegenerative related conditions.

Because of the unpredictability of Cannabis cultivation, the invention requires that all extract preparations of cannabinoid(s) be analyzed to determine the precise concentrations of relevant cannabinoids, especially THCA, THC, CBDA, CBD and CBG for use in preparing unit dosage forms of the invention.

### Pharmaco-Analytical Testing Of Cannabinoid(S) for Use In Preparation Of the Defined Dose Oral Combination

Any chemical analytical method may be employed to determine the amount of the cannabinoids in the preparation used for formulating the UDF. Many methods are available to those skilled in the art, such as those found in Thomas, B F and E l Sohly, M 2015 “The Analytical Chemistry of Cannabis: Quality Assessment, Assurance, and Regulation of Medicinal Marijuana and Cannabinoid Preparations” (Elsevier). See also Wang et al. (2016) Decarboxylation Study of Acidic Cannabinoids: A Novel Approach Using Ultra-High-Performance Supercritical Fluid Chromatography/Photodiode Array-Mass Spectrometry. Cannabis Cannabinoid Res.; 1(1): 262-271; and Wang et al. (2017) Quantitative Determination of Cannabinoids in Cannabis and Cannabis Products Using Ultra-High-Performance Supercritical Fluid Chromatography and Diode Array/Mass Spectrometric Detection. J Forensic Sci.; 62(3):602-611.) A particularly recommended approach is found at Mudge et al. (2017) Anal Bioanal Chem (2017) 409:3153-3163 DOI 10.1007/s00216-017-0256-3.

Testing may be performed to identify the cannabinoid content of the ground dried plant form, any other solid form or a liquid extract preparation.

Testing may be required at one step or at multiple steps in the production process. It may be first performed as a batch assay to ascertain amounts of relevant cannabinoids from a particular harvest or extraction process. The representative sample and measurement technique must be sufficient to represent all samples of the process batch within the degree of variability tolerated by the overall process, namely +/−25% of the defined dose of each cannabinoid. Depending on the result, the cannabinoid preparation may need to be adjusted (either diluted or concentrated) to generate a cannabinoid preparation to meet the tolerance range of volume/dose range for manufacturing specifications of the UDF. The operator will have available a variety of cannabinoid diluents or concentrating processes and/or oils of known cannabinoid concentrations to adjust the preparation. Often only one cannabinoid will need to be added, the others being already at satisfactory levels. The operator can determine by simple algorithm which amount of which additives and/or which concentration steps are required to obtain the desired preparation. The final preparation of cannabinoid may again be chemically analyzed. Any final preparation which is not within tolerance levels is discarded or re-processed until desired cannabinoid levels are obtained. The final tolerance level is within +/−25%, preferably within +/−20%, +/−15%, +/−10%, +/−5%, +/−2% and most preferably within +/−about 1% of the desired in-going amount of each defined dose cannabinoid in the preparation used for formulating the UDF. Alternatively stated, the UDF is expected to contain a dose of from 80% to 120% of the amount stated on product label. Preferably the range will be significantly more precise.

Where the method of the invention requires a Cannabis grinding step, this step must be executed properly to achieve the defined dose of the invention. Grinding risks degradation of the product by generation of heat, by clumping of sticky materials, and by loss of material to the grinding instrument. All aspects must be carefully controlled to achieve superior results.

Preferably, Cannabis will be ground to sieve through a mesh of not larger than about 0.1 mm to about 3 mm, or any 0.1 mm increment therebetween, more preferably not larger than about 1.5, mm in any surface dimension. In some embodiments, the sieve comprises 30, 60, or 120 mesh. In some embodiments, the sieve comprises an average opening size of about 0.595 mm, about 0.250 mm, or about 0.125 mm. Cannabis material may include, without limitation, the leaves, inflorescences, flowers, or buds of one or more Cannabis plants. The grinding step may use any grinding method or methods, such as hand grinding, machine grinding, or use of a chipper or mulcher, provided that a consistent milled size product as homogenous as possible is generated without degradation. Degradation can occur through generation of heat during the grinding process and should be carefully controlled.

### Biosynthetic Production of Cannabinoids

Biosynthesis of cannabinoids by engineered microbial strains (e.g. using eukaryotes, including but not limited to Yeast, Pichia, microalgae, or plant cell-based systems; or prokaryotes including but not limited to E. coli) is an alternative strategy for the production of cannabinoids. The identification of the enzymes involved in cannabinoid biosynthetic pathways enables the reconstruction of the pathway using a suitable heterologous host system. In addition, enzymes can be reconstituted in a cell-extract or a cell-free system to generate cannabinoids from precursor molecules. A synthetic biology approach can be especially interesting for the production of less-abundant cannabinoids. A wide variety of biosynthetic pathways for cannabinoids are set out in Carvahlo et al. (2017) FEMS Yeast Research, 17, 2017, fox037 doi: 10.1093/femsyr/fox037.

### Analytical Identification of Terpenes and Other Cannabis Plant Components in the Cannabinoid Preparation

Depending on the extraction process employed, a variety of other plant constituents may be extracted from Cannabis along with the cannabinoids. It may be desirable to identify and confirm concentrations of these components. Terpenes, chlorophylls, other alkaloids and macromolecules may also be detected by gas chromatography, mass spectroscopy, high-pressure liquid chromatography, or techniques standard in the art.

In certain embodiments, the signifier used with the unit dosage form product may also indicate the defined dose of such additional plant components.

Additional NHPs: Certain embodiments of the invention incorporate an additional natural health product or dietary supplement, or an alternate form of Boswellia extract. A wide range of such products may be included. They may provide further surprising and synergistic advantages to the composition, or they may simply enhance the product subjectively or objectively. While any NHP or dietary supplement that is safe for human consumption at the dosage provided could be employed, most preferred for the invention are selected from among one or more of: Curcumin and/or turmeric, Palmitoylethanolamine (PEA), DL-Phenylalanine (DLPA), Gamma aminobutyric acid (GABA), Acetyl-L-carnitine (ALC), Alpha lipoic acid (ALA), 5-hydroxytryptophan (5-HTP), Echinicaea, Lavender, and Melatonin. Further alternatives include Ashwagandha (root), St. John's Wort Extract (aerial), Valerian (root), Rhodiola Rosea Extract (root), Lemon Balm Extract (leaf), L-Theanine, Passion Flower (herb), cyracos, gotu kola, chamomile, skullcap, roseroot, ginkgo, Iranian borage, milk thistle, bitter orange, sage, L-lysine, L-arginine, Hops, Green Tea, calcium-magnesium, Vitamin A (beta carotene), Magnolia officinalis, Vitamin D3, Pyridoxal-5-phosphate (P5P), St Johns wort, Cayenne, pepper, wasabi, evening primrose, Arnica Oil, Ephedra, White Willow, Ginger, Cinnamon, Peppermint Oil, Thiamin (Vitamin B1) (as thiamin mononitrate), Riboflavin (Vitamin B2), Niacin (Vitamin B3) (as nicotinamide), Vitamin B6 (pyridoxine HCl ), Vitamin B12 (cyanocobalamin), California Poppy, Mullein Verbascum thapsus (L.), Kava Piper methysticum (G. Forst.), Linden Tilia cordata (Mill.), Catnip Nepeta cataria (L.), Magnesium, D-Ribose, Rhodiola Rosea, caffine, Branched-Chain Amino Acids Wheatgrass Shot, Cordyceps, Schisandra Berry, Siberian Ginseng (Eleuthero root), Yerba Mate Tea, Spirulina, Maca Root, Reishi Mushroom, Probiotics, Astragalus, He Shou Wu (Fallopia multiflora or polygonum multiflorum), Cola acuminata (Kola nut), Vitamin C, Centella asiatica (Gotu kola), L-tryosine, Glycine, Pinine, Alpha-pinene, SAMe, DHEA, Co enzyme q10 and glutathione. The additional NHP may also be selected from among the Essential Oils: Anise (Pimpinella anisum (L.)), Basil (Ocimum basilicum (L.)), Bay (Laurus nobilis (L.)), Bergamot (Citrus aurantium var. bergamia (Risso)), Chamomile (German) (Matricaria recutita (L.)), Chamomile (Roman) (Chamaemelum nobile (L.) All.), Coriander (Coriandrum sativum (L.)), Lavender (Lavandula angustifolia (Mill.)), Neroli (Citrus aurantium (L.) var. amara), Rose (Rosa damascena (Mill.)), Sandalwood (Santalum album (L.)), Thyme (Thymus vulgaris (L.)), Vetiver (Vetiveria zizanioides (Nash),) Yarrow (Achillea millefolium (L.)), and Ylang ylang (Cananga odorata (Lam.) var. genuine).

The oral formulation of the invention may optionally further comprise additional components such as but not limited to carrier oils, surfactants, stabilizers, antioxidants, preservatives and excipients, as further described below.

A wide variety of carrier oils may be employed to dissolve, solubilize or otherwise formulate the components of the invention into a liquid or semi-solid formulation suitable for manufacturing the oral formulation and unit dosage forms of the invention. Carrier oils may comprise short chain, medium chain and/or long chain fatty acids. Typically, carrier oils comprise, by mass, from about 1% to about 99%, about 5% to about 93%, about 25% to about 85%, and optionally about 5% to about 35% of the UDF. The oils may be formulated with the cannabinoid and/or the NHP components of the invention through any known formulation process, including but not limited to oil-in-water emulsions, liposomes (e.g. fully encapsulated or aggregated), and nanoparticles. Omega-3, omega-6 and w-9 fatty acids are desirable. In some embodiments, the carrier oils comprise ratios of omega-3 oils to omega-6 oils (on a weight per weight basis) of 1.0 or higher, including ratios of 1.5, 2.0, 2.2, and 3.75. Omega-3 oils include essential oils such as EPA, DHA and alpha lipoic acid. The carrier oils are preferably extracts of plants or plant parts such as nuts, berries, roots, flowers of plants. All carrier oils employed will be safe for human consumption at the dosages provided. For use in a softgel or hardgel of the invention, oil-based preparations may be mixed with a surfactant, such as but not limited to Labrasol™. Surfactants, typically added at about 1-10% by weight, allow the formulation to convert to an emulsion upon exposure to the aqueous environment of the gut. Such formulations are sometimes called self-emulsifying drug delivery systems (SEDDS). Emulsions can be useful to enhance bioavailability of active ingredients.

The term “antioxidant' is used herein includes any compound or combination of compounds that prevent or slow down oxidation of components caused by the damaging reactive oxygen species (ROS). Any of the known antioxidants may be used, including but not limited to tocopherols, phospholipids (PL), phytosterols, phycocyanin, vitamins E, A and C, betacarotene, coenzyme Q10, fatty acids omega-3, omega-6 and w-9, phytoantioxidants such as polyphenols, terpenes as butylated hydroxyanisole (BHA), butylated hydroxytoluene (BHT), propyl gallate, lecithin, sesamin, sesamol, sesamolin, α-tocopherol, γ-tocopherol, salicylic acid, ascorbic acid, ascorbyl palmitate, fumaric acid, malic acid, sodium ascorbate and sodium meta-bisulphite, as well as chelating agents such as disodium EDTA. Pharmaceutically acceptable nutraceutical dietary supplements may also be employed as anti-oxidants including plants, alga, and lichen and may include one or more extracts of honeybee propolis, red clover, soybean, caper, almond, milk thistle, green tea, pomegranate, orange red, grape seed, bilberry, fo-ti root, ginseng, English ivy, red algae, brown algae, green algae and lichens.

Selection of excipients for the unit dosage form is a skill well known to those in the art of pharmaceutical dosage forms. Excipients may include one or more pharmaceutically acceptable carriers, diluents, fillers, hinders, lubricants, glidants, disintegrants, bulking agents, flavourants or any combination thereof. Non-limiting examples of suitable pharmaceutically acceptable carriers, diluents or fillers for use in the invention include lactose (for example, spray-dried lactose, .alpha.-lactose, .beta.-lactose), or other commercially available forms of lactose, lactitol, saccharose, sorbitol, mannitol, dextrates, dextrins, dextrose, maltodextrin, croscarmellose sodium, microcrystalline cellulose (for example, microcrystalline cellulose available under the trade mark Avicel), hydroxypropylcellulose, L-hydroxypropylcellulose (low substituted), hydroxypropyl methylcellulose (HPMC), methylcellulose polymers (such as, for example, Methocel A, Methocel A4C, Methocel A15C, Methocel A4M), hydroxyethylcellulose, sodium carboxymethylcellulose, carboxymethylene, carboxymethyl hydroxyethylcellulose and other cellulose derivatives, pre-gelatinized starch, starches or modified starches (including potato starch, corn starch, maize starch and rice starch) and the like. Typically glidants and lubricants may also be included in the invention. Non-limiting examples include stearic acid and pharmaceutically acceptable salts or esters thereof (for example, magnesium stearate, calcium stearate, sodium stearyl fumarate or other metallic stearate), talc, waxes (for example, microcrystalline waxes) and glycerides, light mineral oil, PEG, silica acid or a derivative or salt thereof (for example, silicates, silicon dioxide, colloidal silicon dioxide and polymers thereof, crospovidone, magnesium aluminosilicate and/or magnesium alumina metasilicate), sucrose ester of fatty acids, hydrogenated vegetable oils (for example, hydrogenated castor oil), or mixtures thereof or any other suitable lubricant. Suitably one or more binders may also be present in the invention and non-limiting examples of suitable binders are, for example, polyvinyl pyrrolidone (also known as povidone), polyethylene glycol(s), acacia, alginic acid, agar, calcium carragenan, cellulose derivatives such as ethyl cellulose, methyl cellulose, hydroxypropyl cellulose, hydroxypropyl methyl cellulose, sodium carboxymethylcellulose, dextrin, gelatin, gum arabic, guar gum, tragacanth, sodium alginate, or mixtures thereof or any other suitable binder. Suitable disintegrants may also be present in the invention. Examples include, but are not limited to, hydroxylpropyl cellulose (HPC), low density HPC, carboxymethylcellulose (CMC), sodium CMC, calcium CMC, croscarmellose sodium; starches exemplified under examples of fillers and also carboxymethyl starch, hydroxylpropyl starch, modified starch; crystalline cellulose, sodium starch glycolate; alginic acid or a salt thereof, such as sodium alginate or their equivalents and any combination thereof.

The total moisture (water) content of the UDF must be selected to ensure appropriate stability and shelf-life for the product. Those skilled in the art are able to identify acceptable ranges depending on the form of UDF selected. Softgels are particularly sensitive to water content as water will weaken and dissolve softgel gelatin capsules. Water content is typically kept below 30% and preferably below 5% of the total mass.

In preferred embodiments, certain potential contaminants are eliminated, avoided, or present at trace levels considered acceptable for human consumption. In particular, the preferred embodiments eliminate, avoid or reduce the presence of organic solvents, pest control products, di-acetyl and ammonia.

Organic solvents: In a preferred embodiment, organic solvent used to extract the cannabinoid and/or Boswellia extract is largely removed from the preparation before formulation in the UDF. Solvent may be removed by evaporation or other known technique. In all preferred embodiments the level of residual solvent is acceptable under ICH guideline topic Q3C(R5). The objective of this guideline is to recommend acceptable amounts for residual solvents in pharmaceuticals for the safety of the patient. The guideline recommends use of less toxic solvents and describes levels considered to be toxicologically acceptable for some residual solvents.

Trace pest control product: In a preferred embodiment any pest control product used in the cultivation of Cannabis or the Boswellia extract, and any derivatives thereof, are removed before combination of the components in the UDF. If such pest control products cannot be fully removed, they preferably do not exceed any maximum residue limit specified for the pest control product, its components or derivatives under the Pest Control Products Act (Canada), or the corresponding act in the relevant country.

Oral formulations of the invention may be further improved by eliminating and ensuring undetectable levels of contaminants that are negatively associated with Cannabis consumption. For example, preferred embodiments of the invention comprise no detectable levels of di-acetyl (CH₃CO)₂, also called 2,3-butanedione, an additive sometimes used in preparations of Cannabis for smoking/vaping. Also preferred is no detectable level of ammonia, which may contaminate the source Cannabis crop due to over-fertilization and lack of flushing during hydroponic cultivation.

In a preferred embodiment, the UDF meets the requirements of a dissolution or disintegration test that is applicable to its formulation and that is set out in European Pharmacopoeia, The Canadian Formulary, The United States Pharmacopoeia, and/or The Pharmaceutical Codex: Principles and Practices of Pharmaceuticals.

### General UDF Production Methods

Having selected the amounts and concentrations of all ingredients of the oral formulation of the invention, the ingredients will be formulated together for preparing the unit dosage form. Those skilled in the art are familiar with identifying preferred formulation techniques for the UDF. In a preferred embodiment, the UDF is a pill, tablet, capsule, film, or wafer, any of which may optionally be orally disintegrating, or a lollipop, lozenge, oil, tincture, or syrup. The formulation process will be adjusted accordingly. Pills and tablets are prepared from solid formulations. Syrups, oils and tincture are liquid formulations. An orally disintegrating film, wafer, tablet or a lollipop or lozenge provides the UDF in an oral form wherein the active ingredients are at least partly absorbed directly in the buccal cavity. Capsules may be either solid formulations (e.g. powders or particles in a hard-gel) or liquid formulations (e.g. oil-based formulations used in soft-gels). Oil based formulations with little or no water are typically easily encapsulated. Such oil-based preparations may be mixed with a surfactant, such as but not limited to Labrasol™. Oil-in-water formulations may comprise microemulsions, liposomes, nanoemulsions and other forms known in the art.

NHP component may be physically separated from cannabinoid, or the two components may be mixed together. Physical separation by particles (which do not mix) or by capsule-within-capsule design. Mixing together can be achieved by formulation in the same liquid carrier, or by mixing of powders/particulates before capsule loading. An oil-in-water type emulsion, and other variants where the components may be separated at molecular level by hydrophilicity is considered “mixed together”, in the sense that cannabinoids and NHPs are evenly dispersed throughout the entire capsule UDF.

Preferred capsule types are soft gelatin capsules (softgels) and hard gelatin capsules. Soft Gelatin Capsules (softgels) are well known in the art. Typically soft-gels are used for formulations not based on water, such as oil-based solutions, because water based solutions would dissolve the gelatin. The basic steps of softgel manufacturing are: Gelatin Preparation (the process of blending and heating granulated gelatin into a thick syrup for use in encapsulation); Fill Material Preparation (the process of preparing the non-aqueous oil or paste containing the NHP and cannabinoid components that will be encapsulated); Encapsulation (the process of converting the gel mass into a thin layer of gelatin and wrapping it around the fill material to form a softgel); Drying (the process which removes excess moisture from the gelatin shell to shrink and firm up the softgel); the softgel could incorporate a Coating step (the process of coating the capsule with a coating designed to release the capsule within the digestive system); and finally Cleaning, Inspection and Sorting. Automated or semi-automated manufacturing of softgels and can be achieved using commercially available equipment, such as that provided by CapPlus Technologies, SaintyCo, and many others.

Hard gelatin capsules are made of two parts, the body and a cap. This form of capsule holds dry ingredients in the form of powders, granules or tiny pellets. They may also include Cannabis oils of various viscosity, such as diluted Cannabis oil and concentrated Cannabis extracts. The body is first filled with the mix of active ingredients and any excipients used, and then closed with the cap using either a manual or automated or semi-automated capsule filling machine, such as those commercially available from Bosch, Zanazzi, etc. Banding of hard gelatin capsules is sometimes useful to prevent leakage.

A wide range of capsule sizes are suitable for use with the invention. A UDF in capsule form may be any size suitable for human swallowing and for example may be selected from among any of the standard commercial capsule sizes, and/or may be selected from among about 100 mg, 200 mg, 250 mg, 300 mg, 400 mg, 500 mg, 600 mg, 700 mg, 750 mg, 800 mg, 900 mg, 1000 mg or any about 10 mg interval between 0 mg and 1000 mg.

The inventors recognize that advantages may be achieved by use of a dose form that is substantially opaque to one or both of ultraviolet and visible light, such as a photo- and/or UV-opaque gelatin capsule. A general form of this technology is described in co-owned patent application U.S. Ser. No. 62/837,848 filed 24 Apr. 2019 and incorporated herein by reference.

Delayed release to the gastrointestinal track can be achieved for softgels or hard gels by enteric coatings which delay disintegration until after passing from stomach to the intestine; or by formulation techniques such as pellets which resist release until they pass into a specific intestinal domain. Such techniques are widely known in the art. An example is WIPO patent publication WO2017075215A1 to McGuffy and Bell for extended release film-coated softgel or hard-shell capsules.

A wide variety of technologies are available for a buccal or sublingual formulation such as an orally disintegrating thin film, wafer or tablet, or a lollipop, and/or lozenge. Sublingual tablets, wafers, films and strips can be designed to rapidly disintegrate (5-15 seconds) providing rapid access to buccal cavity capillaries and avoid the hostile environment of the gastrointestinal track. Lollipops and lozenges provide a combination of buccal and gastric administration. The technologies are widely used with therapeutic agents where rapid onset is desired. (See Lamey and Lewis “Buccal and Sublingual Delivery of Drugs” Ch 2 in “Routes of Drug Administration” Ed. Florence and Salole (Butterworth-Heinemann)).

### Association With Signifier

The UDF of the invention preferably comprises a signifier which allows the consumer to determine the defined dose of selected cannabinoids therein. A “signifier” means a mark, symbol, indicia, striation or the like which may be perceived visually or by touch, which provides information to a consumer about the UDF's specific defined dose. The signifier chosen may have elements of meaning, such as a number and unit, (e.g. “5 mg” or “10 mg” or simply “5” or “10”) or it may be an abstract signifier, where its meaning, in terms of defined dose, can be determined by reference to a standard. The meaning may be determined directly by the consumer or indirectly via a device.

The signifier may be associated directly with the UDF after encapsulation by such means as embossing, or by colour, pattern or shape feature. Alternatively, the signifier may be associated with the packaging. The packaging may include signifiers directly interpreted by consumers or signifiers which are machine readable codes. In all embodiments, the signifier allows the consumer to determine the defined dose of selected cannabinoid(s) therein and optionally the dose of the NHP and any other constituent.

The signifier may be associated directly with the UDF before, during or after encapsulation by such means as edible ink(s) imprinted on the surface of the capsule, or by embossing, by engraving (such as laser-engraving), or by color, pattern or shape feature. The edible ink applied to the capsule may include shellac from about 10% to about 30% by weight, about 20% to 70% by weight of at least one solvent, and at least one soluble or insoluble pigment from about 10% wt to about 40% wt. The shellac provides structure, enhances adherence to the printing plate and capsule, and acts as a pigment carrier. An edible ink formulation may include 10% wt to about 30% wt shellac.

### Packaging

After a UDF is manufactured, storage and delivery to consumer may be provided by:
a. Packaging the UDF individually in a blister pack; or
b. Packaging multiple UDFs in a re-sealable package.

The UDF is preferably provided in a sealed package, which functions as a barrier limiting moisture fluctuation, reducing oxidation, and enhancing shelf-life, etc. The packaging is optionally a gas-impermeable container having a hermetic closure which in the context of the present invention includes a blister pack. The UDFs may be individually sealed and packaged in blister packs. The blister packs may be designed to be child resistant and/or senior friendly in order to increase safety and convenience. While physically protecting the matrix units, the blister pack controls humidity and is impermeable to gas exchange thereby enhancing shelf life.

Examples of the substantially gas exchange impermeable packaging include, but are not limited to, A1/A1 blister, and A1-polychloro-3-fluoroethylene homopolymer/PVC laminate blister. Alternatively, the sealed package may be a re-sealable multi-package impermeable to gas exchange.

UDFs of the invention may be expelled from production into the open blister cavities. Cavity depth and shape must be suitable for the unit. The open blister cavity is then sealed with a gas impermeable membrane to maintain quality of product and to reduce dehydration, rehydration or oxidation. To eliminate oxidation altogether, the packaging may be performed in an inert gas atmosphere. Optionally the blister is packed in an inert gas atmosphere such as nitrogen gas comprising little or no oxygen. To achieve this objective, the final sealing step of the packaging method may be operated in the inert gas atmosphere in a gas enclosure protected from ambient air.

### EXAMPLES

### Example 1: Bioinformatics

### Synergistic Action of Combinations on Gene Expression and Protein Activity Pathways

The inventors have employed a variety of bioinformatics tools to identify the biochemical synergies of the oral combinations proposed herein and to predict their therapeutic effects.

### Bioinformatic Tools

Gene Expression Overlaps: Gene expression overlaps were identified from the Comparative Toxicogenomics Database (CTD), MDI Biological Laboratory, Salisbury Cove, Maine, and NC State University, Raleigh, North Carolina. World Wide Web (URL: http://ctdbase.org/). (see Davis AP et al. The Comparative Toxicogenomics Database: update 2017. Nucleic Acids Res. 2016 Sep. 19.

NHP-Protein Interactions: NHP-protein interactions are taken from STITCH (search tool for interactions of chemicals'), a bioinformatics tool available at http://stitch.embl.de. STITCH lists known chemical-protein interactions and integrates information about interactions from metabolic pathways, crystal structures, binding experiments and drug-target relationships. (Sklarczyk et al. (2015) STITCH 5: augmenting protein-chemical interaction networks with tissue and affinity data. NAR 2016 (44) D380-D384.) STITCH has been used by the inventors to investigate the shared pathways activated by the individual product components.

Genevenn and Enrichr programs have been employed for studying gene expression patterns induced by exposure of cells to active agents. Genevenn (http://genevenn.sourceforge.net) finds gene expression overlaps. Enrichr (http://amp.pharm.mssm.edu/Enrichr/) was applied to the gene overlap to search through libraries of data (disease associations, expression data, biochemical databases, etc) that matches the gene/protein to the overlapping pathways and that way identified the gene expression pathways. Enrichr identified cell signaling pathways for the overlapping genes. Enrichr is named for the function of the gene lists/terms that are enriched cell lines that express the receptors.

### Assessment of Boswellia Extract and Selected Cannabinoids

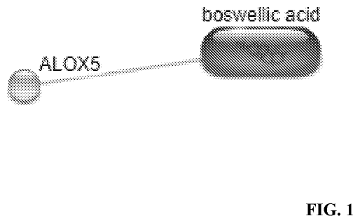

At a biochemical level, Boswellia extract is known to directly interact with human metabolic pathway illustrated in FIG. 1. The pathway is ALOX5, referring to arachidonate 5-lipoxygenase which catalyzes the first step in leukotriene biosynthesis and thereby plays a role in inflammatory processes.

Using bioinformatic tools the inventors have also identified gene expression effects induced by Boswellia extract. Using the STITCH database (Szklarczyk et al 2015), 11-keto-β-boswellic acid (AKBA) was found to target Arachidonate 5-lipoxygenase (ALOX5) gene expression pathway which is necessary for 5-lipoxygenase and therefore for the production of leukotrienes which are lipid mediators of inflammation. Leukotrienes, potent chemo-attractants for neutrophils, are released by activated neutrophils following oxygenation of arachidonic acid by the enzyme 5-LOX (Ford-Hutchinson 1990).

Separately, two overlapping gene expression pathways between Boswellic acid and THC were identified as PPP2CA and PPP3CB.

As described below, recognizing these effects, the inventors have combined Boswellia extract with the now identified selected cannabinoids where corresponding effects on shared underlying pathways not previously recognized lead to the synergistic and surprising results of the invention.

### Example 2: In Vitro Biological Examples

Amounts and concentrations of Boswellia extract and the selected cannabinoid for testing in the assays below (both in vitro and in vivo) are chosen to correspond to the dose that would be expected upon administration to a human of the oral formulation or one or more unit dosage forms of the invention taken at the same time. For example, in cell-based assays the amounts are adjusted to correspond to present the cells with the expected physiological level that would be encountered in a human consuming an oral formulation of the invention. Similarly, in animal models, the amount tested is adjusted from the UDF used in humans to a corresponding ratio in the animal based on mg/kg, factoring in expected oral uptake and absorption differences. Those skilled in the art are familiar with defining and selecting the concentrations to be tested in the assays and extrapolating back to the appropriate dosage for the UDF in humans.

### Background for Identification of Synergies from Cell-based and Biochemical Assays

### Synergy Index

The use of multiple therapeutic agents may target multiple targets and/or multiple diseases simultaneously. The use of agents with similar mechanisms or modes of action may also maximize the effect against single target or a disease and treat it more effectively. In addition, coordinated action at multiple molecular targets can provide unique therapeutic benefit not achievable with the “one-drug, one-target” paradigm.

Two or more therapeutic agents that individually produce overtly similar effects will sometimes display greatly enhanced effects when given in combination. When the combined effect is greater than that predicted by their individual potencies, the combination is described as synergistic, and more specifically a positive synergy. A synergistic interaction allows the use of lower sub-therapeutic doses of the combination constituents, a situation that may reduce potential adverse reactions. Sometimes a positive synergy may conceptually be stated as a “1+1=3” effect.

The possible favorable outcomes for synergism include, but are not limited to:
a. Increasing the efficacy of the beneficial therapeutic effect
b. Decreasing the dosage but increasing or maintaining the same efficacy to reduce cost and avoid undesirable adverse effects
c. Minimizing or slowing down the development of drug resistance, and
d. Providing selective synergism against target (or efficacy synergism) versus host (or toxicity antagonism)

Evaluation of synergistic effects for cannabinoid and NHP combinations can be evaluated in cell based and biochemical receptor binding assays, by determining effects over a range of ratios and concentrations and analyzed by CalcuSyn software program (Biosoft, Ferguson, Mo., USA). This program could be used for dose effect analysis for single agents using the median-effect equation and for agents in combination using both the median-effect equation and the combination index equation (Chou and Talalay, 1984, Chou and Hayball, 1996, Chou and Martin, 2005 and Chou, 2006). The occurrence of ratio-dependent synergy is determined by plotting the combination index (CI<1, synergy (or positive synergy); CI˜1, additivity; and CI>1, antagonism (or negative synergy) versus the fraction of cells affected (Fa), which indirectly reflects the therapeutic agent concentration.

### In Vitro Cell-Based Assays

### 1. NF-κB Luciferase Assay

NF-κB (Nuclear Factor-Kappa B) signaling network is the main pathway that plays a crucial role influencing a broad range of biological processes including innate and adaptive immunity, inflammation and stress responses. It could be activated by infection, stress, diet, chemotherapy, obesity, addiction as well as by a variety of pro-inflammatory cytokines such as IL-1β, TNF-α, IL-6, IL-8.

The NF-kB pathway inhibition in response to treatment with the Cannabinoid and NHP either as single agents or in combination will be monitored using the Luciferase Reporter gene assay described in Del Prete et al, J. Nat. Prod. 2017, 80, 2276-2283.

For the anti-NF-κB activity stably transfected NIH-3T3-KBFLuc cells are treated with different concentration of NHP compounds for 30 minutes and then stimulated with TNF-α (30 ng/mL). After treatment, the cells are washed twice in phosphate-buffered saline and lysed in 25 mM Tris-phosphate pH 7.8, 8 mM MgCl2, 1 mM DTT, 1% Triton X-100, and 7% glycerol during 15 min at room temperature in a horizontal shaker. Luciferase activity in the supernatant is measured using a TriStar2 Berthold/LB942 multimode reader (Berthold Technologies) following the manufacturer's instructions of the luciferase assay kit (Promega, Madison, Wis., US). For NF-κB inhibition the RLU (relative light units) is calculated, and the results are normalized to 100% stimulation induced by induced by TNF-α (100% activation). The results are presented from three independent experiments.

### 2. CB₁R Cannabinoid Receptor Agonist and Antagonist Assays

The CB₁R and CB₂R agonistic and antagonistic activities in response to treatment with the Cannabinoid and NHP either as single agents or in combination are measured using the HEK293T-CB₁R and HEK293T-CB₂R cells lines stably transfected with human CB1R and CB2R cDNA respectively. Briefly, HEK293T-CB1 cells are transiently transfected with 0.2 μg of the reporter plasmid CRE-luc that contains six consensus cAMP responsive elements (CRE) linked to firefly luciferase reporter gene using Roti-Fect (Carl Roth, Karlsruhe, Germany) following manufacturer's instructions. The increase in cAMP levels activates the pCRE-Luc system, inducing the expression of the luciferase reporter gene.

For CB1R agonistic activity, the transfected HEK293T0CB1-CRE-luc cells are treated with a range of concentrations of the compounds. For CB1R antagonistic and allosterism activity, these cells are incubated with different concentrations of the compounds for 30 minutes and then treated with the CB1R agonist CP-55940. Forskolin, an adenylate cyclase activator, is used at 10 μM along as a positive control of cAMP signaling pathway activated by a CB1R-independent mechanism; Cp-55940, a CB1R agonist, is used at 1 μM alone as a positive control of cAMP signaling pathway activated by a CB1R-dependent mechanism.

After 6 hours of stimulation the cells are washed twice in phosphate-buffered saline and lysed in 25 mM Tris-phosphate pH 7.8, 8 mM MgCl2, 1 mM DTT, 1% Triton X-100, and 7% glycerol during 15 min at room temperature in a horizontal shaker. After centrifugation, luciferase activity in the supernatant is measured using a TriStar2 Berthold/LB942 multimode reader (Berthold Technologies) following the instructions of the luciferase assay kit (Promega, Madison, Wis., USA). The results are represented as the mean of at least five independent experiments, with the SD lower than 15%.

Thus, as part of the Synergy Index, the combinations of the invention, and the components separately, can be tested for agonist and antagonist behaviour against CB₁R and agonist behaviour against CB₂R. Such assays are widely available commercially. A preferred assay is the Ready-to-Assay™ CB₁R (catalog: HTS019RTA) and Ready-to-Assay™ CB₂R (catalog: HTS020RTA) cell-based assays provided by Millipore.

The combinations of the invention are found to demonstrate surprising and unexpected synergies.

Example 2A: Agonistic activity on CB1 receptor. The agonistic activity of AKBA alone or in combination with THC or THCA on CB1 receptor was analyzed using the HEK293T-CB1 cell line and the CRE-Luc reporter that is sensitive to the increase of cellular cAMP level after CB1R ligand occupation. The CB1R agonistic activity was reflected by the induction of CRE-Luc activity relative and expressed as a fold change over non-stimulated cells.

95% pure powdered AKBA (Sigma Aldrich, # A9855), dissolved in dimethyl sulfoxide (DMSO), was prepared to five different concentration of AKBA alone (5, 10, 25, 50 and 100 μM) or in the presence of 10 μM THC or 10 μM THCA were evaluated for CB1R agonistic activity. Table 2 summarizes obtained data.

TABLE 2
AKBA alone and AKBA combinations CB1R agonistic
activity data. Data are represented as fold change of
CB1R activity. An increase of the fold change
≥ 2.5-fold indicates CB1R agonistic activity. An
increase of the fold change ≥ 4-fold indicates CB1R
positive allosteric activity. Data are from independent
experiments where the positive control (CP55940;
CB1R agonist) showed an induction of CB1R
activity ≥ 2.5-fold increase. Only treatments
that the mean of three consecutive independent
experiments shows a positive result: CB1R
activity ≥ 2.5-fold increase or ≥ 4-fold increase are
considered CB1R agonists or CB1R positive allosteric
modulators, respectively.

| | Experi- | Experi- | Experi- | Conclusions: |
|---|---|---|---|---|
| | ment | ment | ment | Is CB1R |
| | 1 | 2 | 3 | agonist |
| | Fold | Fold | Fold | effect |
| | change | change | change | observed? |
| CP 2.5 μM | 2.5 | 2.5 | 2.5 | + |
| (control agonist) | | | | |
| AKBA 5 μM | 0.8 | 0.8 | 1.1 | Negative |
| AKBA 10 μM | 1.0 | 1.3 | 0.9 | Negative |
| AKBA 25 μM | 1.0 | 1.1 | 0.8 | Negative |
| AKBA 50 μM | 1.3 | 1.3 | 0.7 | Negative |
| AKBA 100 μM | 2.5 | 1.1 | 0.7 | Negative |
| THCA 10 μM | 1.2 | 1.1 | 0.8 | Negative |
| AKBA 5 μM + | 1.2 | 1.0 | 0.8 | Negative |
| THCA 10 μM | | | | |
| AKBA 10 μM + | 1.2 | 1.1 | 0.8 | Negative |
| THCA 10 μM | | | | |
| AKBA 25 μM + | 0.9 | 1.1 | 0.7 | Negative |
| THCA 10 μM | | | | |
| AKBA 50 μM + | 2.4 | 2.1 | 0.9 | Negative |
| THCA 10 μM | | | | |
| AKBA 100 μM + | 3.4 | 2.7 | 1.3 | Negative |
| THCA 10 μM | | | | |
| THC 10 μM | 3.9 | 4.2 | 2.7 | + |
| AKBA 5 μM + | 4.5 | 2.9 | 2.7 | + |
| THC 10 μM | | | | |
| AKBA 10 μM + | 4.3 | 3.5 | 2.6 | + |
| THC 10 μM | | | | |
| AKBA 25 μM + | 5.2 | 5.1 | 4.4 | Positive |
| THC 10 μM | | | | synergy |
| AKBA 50 μM + | 10.9 | 4.6 | 6.0 | Positive |
| THC 10 μM | | | | synergy |
| AKBA 100 μM + | 19.2 | 12.4 | 13.6 | Positive |
| THC 10 μM | | | | synergy |

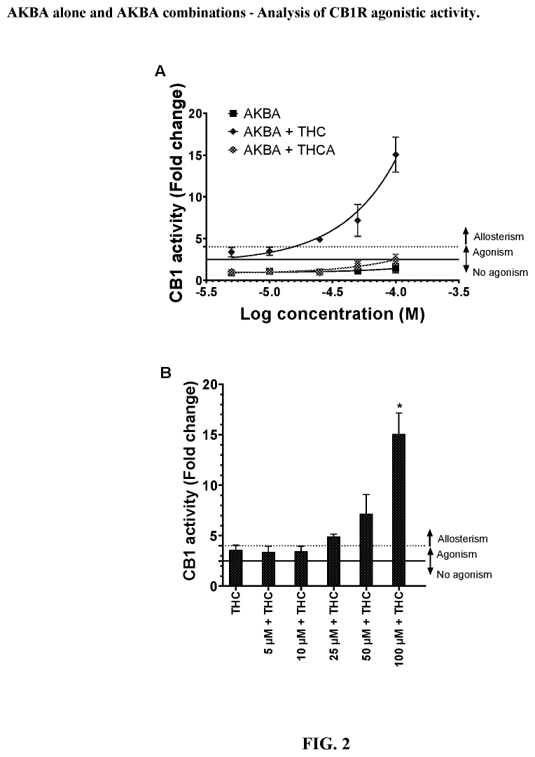

AKBA alone and AKBA-THCA combinations did not show CB1R agonistic activity in any tested concentration. However, THC alone does have CB1R agonist activity. In AKBA-THC combination treatment, the THC-associated CB1R agonistic activity was progressively increased in a potent manner as the concentration of AKBA was increased (FIG. 2A). The EC50 value of AKBA to induce the 10 μM THC CB1R agonistic activity was 69.67±20.54 μM. An additional analysis comparing the effects of THC alone versus AKBA-THC treatments showed a statistically significant difference at 100 μM confirming the positive synergy (allosteric) effect of AKBA on the THC CB1R agonistic activity (FIG. 2B).

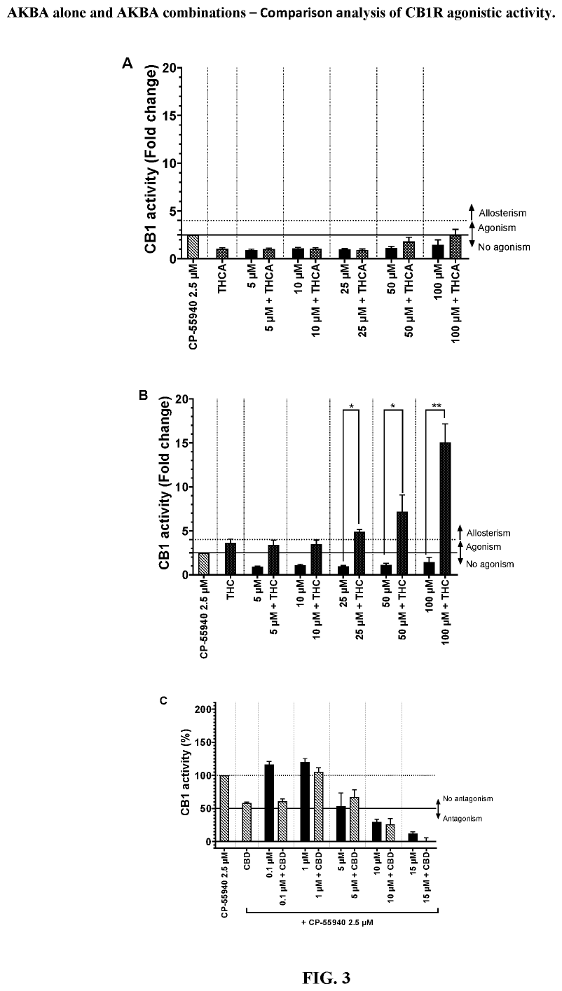

A deeper comparison study of the treatments was performed to further analyze the possible differences between AKBA alone and AKBA combination treatments. This study showed no differences in the effect between any of the tested concentrations of AKBA and AKBA-THCA combo (FIG. 3A). In contrast, this analysis showed that the presence of THC at 25, 50 and 100 μm AKBA concentrations produced a potent increase of the CB1 agonistic activity, which is statistically significant, demonstrating the potent positive synergy (allosteric) behavior of AKBA (FIG. 3B and FIG. 3C).

Example 2B: Antagonistic activity on CB1 receptor. The antagonistic activity of AKBA alone or in combination with THC, CBD or THCA on CB1 receptor was analyzed using the HEK293T-CB1 cell line and the CRE-Luc reporter that is sensitive to the increase of cellular cAMP level after CB1R ligand occupation. The CB1R antagonistic activity was reflected by the inhibition percentage of CP55940-induced CRE-Luc activity, which is considered 100% stimulation, as described in experimental design section.

AKBA alone and AKBA combinations (THC, CBD and THCA) analysis. As in Example 2A, five different concentrations of AKBA alone (5, 10, 25, 50 and 100 μM) or in the presence of 10 μM THC or THCA or, additionally in this case, 1 μM CBD were evaluated on CB1R antagonistic activity. Table 3 summarizes obtained data.

TABLE 3
AKBA alone and AKBA combinations CB1R
antagonistic activity data. Data are
represented as percentage of CB1R activity.
A reduction of the percentage below 50% indicates
CB1R antagonistic activity. Data are from independent
experiments where the positive
control (CP55940; CB1R agonist) showed an
induction of CB1R activity ≥ 2.5-fold increase.
Only treatments that the mean of three
consecutive independent experiments show
a positive result (CB1R activity ≤ 50%) are
considered antagonists of CB1R activity.

| | Experi- | Experi- | Experi- | Conclusions: |
|---|---|---|---|---|
| | ment | ment | ment | Is CB1R |
| | 1 | 2 | 3 | antagonist |
| | % of | % of | % of | effect |
| | activity | activity | activity | observed? |
| CP 2.5 μM | 100.0 | 100.0 | 100.0 | Negative |
| AKBA 5 μM | 129.6 | 122.9 | 102.7 | Negative |
| AKBA 10 μM | 135.6 | 113.5 | 96.4 | Negative |
| AKBA 25 μM | 203.0 | 157.8 | 127.5 | Allosterism |
| AKBA 50 μM | 499.6 | 536.7 | 474.1 | Allosterism |
| AKBA 100 μM | 1022.4 | 673.8 | 484.6 | Allosterism |
| THCA 10 μM | 163.2 | 118.4 | 86.5 | Negative |
| AKBA 5 μM + | 107.8 | 86.1 | 83.6 | Negative |
| THCA 10 μM | | | | |
| AKBA 10 μM + | 100.7 | 93.2 | 100.9 | Negative |
| THCA 10 μM | | | | |
| AKBA 25 μM + | 76.0 | 161.2 | 101.5 | Negative |
| THCA 10 μM | | | | |
| AKBA50 μM + | 130.9 | 372.4 | 177.9 | Allosterism |
| THCA 10 μM | | | | |
| AKBA 100 μM + | 334.3 | 396.6 | 402.5 | Allosterism |
| THCA 10 μM | | | | |
| CBD 1 μM | 66.2 | 70.7 | 69.5 | + |
| AKBA 5 μM + | 60.9 | 46.5 | 46.3 | + |
| CBD 1 μM | | | | |
| AKBA 10 μM + | 66.9 | 91.8 | 31.0 | + |
| CBD 1 μM | | | | |
| AKBA 25 μM + | 78.4 | 45.4 | 45.9 | + |
| CBD 1 μM | | | | |
| AKBA 50 μM + | 160.0 | 178.0 | 137.0 | Allosterism |
| CBD 1 μM | | | | |
| AKBA 100μM + | 318.4 | 163.5 | 121.6 | Allosterism |
| CBD 1 μM | | | | |
| THC 10 μM | 155.3 | 167.3 | 108.0 | Negative |
| AKBA 5 μM + | 179.2 | 114.7 | 108.6 | Negative |
| THC 10 μM | | | | |
| AKBA 10 μM + | 170.2 | 141.0 | 104.8 | Negative |
| THC 10 μM | | | | |
| AKBA 25 μM + | 209.1 | 204.7 | 175.5 | Allosterism |
| THC 10 μM | | | | |
| AKBA 50 μM + | 435.1 | 182.2 | 241.6 | Allosterism |
| THC 10 μM | | | | |
| AKBA 100 μM + | 769.6 | 497.2 | 545.5 | Allosterism |
| THC 10 μM | | | | |

Based on the data for AKBA-THC combination in the CB1 agonism assay, AKBA did not show an antagonistic effect. In contrast, AKBA showed a strong positive allosteric CB1R effect on CP-55940 activity. This phenomenon was observed by the increase of the CB1R activity percentage (>150%) induced by CP55940. To study this effect, data was re-analyzed and expressed as fold change of CB1R activity (Table 4).

TABLE 4
AKBA alone and AKBA combinations CB1R
positive allosteric activity data. Data are
represented as fold change of CB1R activity. An
increase of the fold change ≥ 4-fold
indicates CB1R positive allosteric agonistic
activity. Only treatments that the mean of three
consecutive independent experiments show a
positive result (CB1R activity ≥ 4-fold change)
are considered positive CB1R allosteric agonists.

| | | | | Conclusions: |
|---|---|---|---|---|
| | | | | Is CB1R |
| | Experiment | Experiment | Experiment | antagonist |
| | 1 | 2 | 3 | effect |
| | Fold change | Fold change | Fold change | observed? |
| CP 2.5 μM | 2.5 | 2.5 | 2.5 | Negative |
| AKBA 5 μM | 3.2 | 3.1 | 2.6 | Negative |
| AKBA 10 μM | 3.4 | 2.8 | 2.4 | Negative |
| AKBA 25 μM | 5.1 | 3.9 | 3.2 | Allosterism |
| AKBA 50 μM | 12.5 | 13.4 | 11.9 | Allosterism |
| AKBA 100 μM | 25.6 | 16.8 | 12.1 | Allosterism |
| THCA 10 μM | 4.1 | 3.0 | 2.2 | Negative |
| AKBA 5 μM + | 2.7 | 2.2 | 2.1 | Negative |
| THCA 10 μM | | | | |
| AKBA 10 μM + | 2.5 | 2.3 | 2.5 | Negative |
| THCA 10 μM | | | | |
| AKBA 25 μM + | 1.9 | 4.0 | 2.5 | Negative |
| THCA 10 μM | | | | |
| AKBA 50 μM + | 3.3 | 9.3 | 4.4 | Allosterism |
| THCA 10 μM | | | | |
| AKBA 100 μM + | 8.4 | 9.9 | 10.1 | Allosterism |
| THCA 10 μM | | | | |
| CBD 1 μM | 1.7 | 1.8 | 1.7 | Negative |
| AKBA 5 μM + | 1.5 | 1.2 | 1.2 | Negative |
| CBD 1 μM | | | | |
| AKBA 10 μM + | 1.7 | 2.3 | 0.8 | Negative |
| CBD 1 μM | | | | |
| AKBA 25 μM + | 2.0 | 1.1 | 1.1 | Negative |
| CBD 1 μM | | | | |
| AKBA 50 μM + | 4.0 | 4.4 | 3.4 | Negative |
| CBD 1 μM | | | | |
| AKBA 100 μM + | 8.0 | 4.1 | 3.0 | Allosterism |
| CBD 1 μM | | | | |
| THC 10 μM | 3.9 | 4.2 | 2.7 | Negative |
| AKBA 5 μM + | 4.5 | 2.9 | 2.7 | Negative |
| THC 10 μM | | | | |
| AKBA 10 μM + | 4.3 | 3.5 | 2.6 | Negative |
| THC 10 μM | | | | |
| AKBA 25 μM + | 5.2 | 5.1 | 4.4 | Allosterism |
| THC10 μM | | | | |
| AKBA 50 μM + | 10.9 | 4.6 | 6.0 | Allosterism |
| THC 10 μM | | | | |
| AKBA 100 μM + | 19.2 | 12.4 | 13.6 | Allosterism |
| THC 10 μM | | | | |

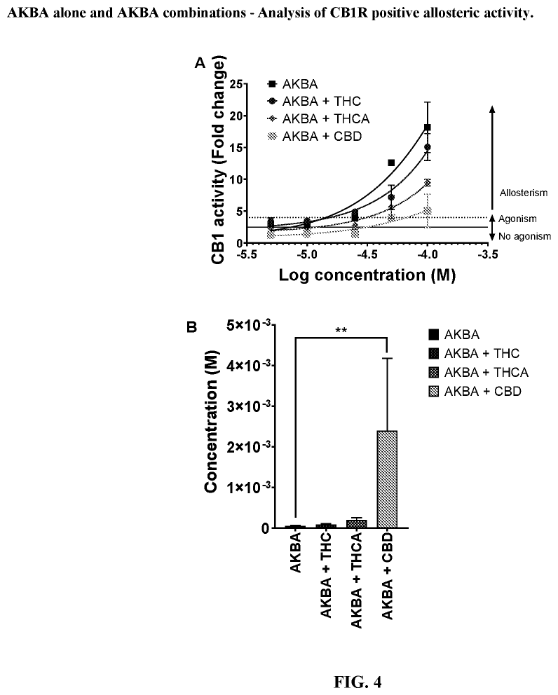

The CB1R positive allosteric effect of AKBA on CP-55940 activity was clear in all tested treatments (FIG. 4A). The AKBA positive allosteric activity EC50 value was 51.71±13.63 μM. In the AKBA-THC and AKBA-THCA combinations a reduction in the potency of the allosteric effect was observed, (AKBA-THC IC50=86.66±23.27 μM and AKBA-THCA IC50=200.08±52.62 μM). The AKBA-CBD combo treatment showed a statistically significant reduction of the AKBA CB1R positive allosteric activity with the consequent increase of the EC50 (**, p<0.05. AKBA-CBD EC50=239.60±81.80 μM) (FIG. 4B).

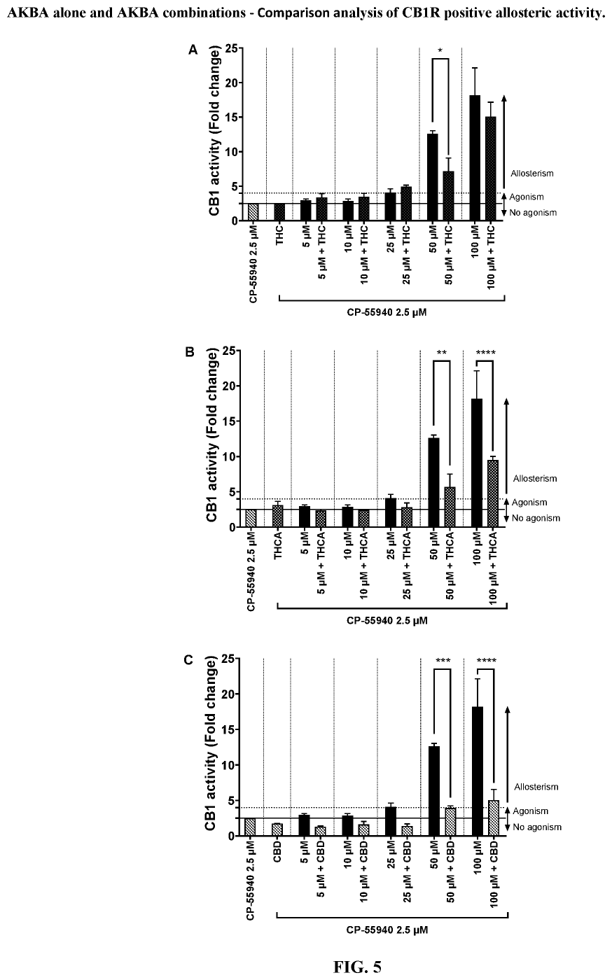

A deeper comparison study of the treatments was performed to further analyzed the possible differences between AKBA alone and AKBA combination treatments. THC did not modify the AKBA effect in any of the tested concentrations (FIG. 5A). In contrast, the presence of THCA reduced the AKBA allosteric activity at 50 and 100 μM (FIG. 5B). And finally, the strongest effect was observed in the presence of CBD at 50 and 100 μM where the allosteric effect of AKBA was almost abolished (FIG. 5C).

Summarized otherwise, the invention can be described as a method to enhance the effect of THC to a consumer by providing it in combination with Boswellia extract. Another description is a “high-impact THC capsule” where a lower dose THC capsule according to the invention has the physiological effect of a higher dose prior art capsule. In some embodiments, the invention provides a physiological impact of THC on the user of about 20% higher than the same dose of THC given without Boswellia extract. In some embodiments, the synergy generated by Boswellia extract provides a physiological impact of THC that is 120%, 150%, 180%, 200%, 250%, 400%, 500% or 750% higher than the same THC dose without Boswellia extract. In other formulations, the dose of THC may be lowered to a fraction of a standard dose, yet still provide the same physiological impact to the user as a higher dose. As such, the invention particularly includes high-impact THC/Boswellia extract capsules having 1 mg, 2 mg, 2.5 mg, 5 mg or 10 mg THC.

### Example 3: Animal Model of PK/PD and Bioavailability

Oral formulations of the invention were tested to determine key pharmacokinetic (PK) parameters and to ensure satisfactory exposure over time. PK assays are used to identify plasma concentration over time, area under the curve (AUC) exposure over 24 hrs, systemic clearance rate (CL) and systemic bioavailability (% F). The combination is also tested against the individual components. The 24 hr exposure identifies if the UDF should be administered QD (once a day) or BID (×2 a day) or more often, or less often.

Standard PK models are widely available and can be performed with a commercial service. A preferred method is to use at least 4 Male Sprague Dawley rats (210-230 g) who receive either an intravenous (i.v. 2, 5, and/or 10 mg/kg) or oral (5, 10 and/or 20 mg/kg) dose of each compound separately, or combined in formulation. Blood, urine, cerebrospinal fluid (CSF) or other appropriate biological fluid is removed at periodic intervals. The biological fluid is tested for active compound(s) in order to construct concentration vs. time profiles. These data are analyzed and pharmacokinetic parameters are calculated in order to assess in vivo pharmacokinetic activity.

The study uses a fixed dose of each component in the combination in a fixed vehicle formulation. In one embodiment PEG (polyethylene-glycol) is an excipient, or alternatively a long chain fatty acid oil carrier. Typically, components are prepared from a powder form, first in 5% ETOH, then with 40% PEG. The components are combined and topped up with distilled water to 100% volume. If components are not completely soluble, PEG may be increased to 60% and also add 10% PG.

By way of example, a PK study may be conducted at 10 mg/kg dose for an oral formulation. 2 mg/kg may be used for IV injection as a comparison. In either case, plasma samples are collected over a 24 hr time-period to determine bioavailability. Plasma samples are tested by HPLC or LC-MS/MS to obtain PK parameters e.g. 3 rats per each route of administration (total n=6) is typically sufficient.

Plasma and other tissue samples are tested for the administered cannabinoids and the Boswellia extract administered to the animal. The samples are also tested for significant metabolites, some of which may have more potent effects than the parent administered compounds. The samples may also be used to determine baseline levels of serum biomarkers which are relevant to the development or treatment of the complex disease models set out further below. Many serum biomarkers are of great interest in the development or treatment of complex disorders. Biomarkers of interest to the compositions of the invention include IL-6, NF-kB, TNF-α, C-reactive protein, and any other biomarker known to be or potentially implicated in the development of a disease or disorder.

### Animal Models of Complex Disease or Condition

Compositions of the invention are tested in models corresponding to the disease and/or conditions proposed for use. These may be selected from among models of anxiety, pain, sleep induction, calmness induction, alertness induction, weight control, weight loss, obesity, diabetes and metabolic syndrome.

For any of the animal assays herein (including human testing), successful treatment may be identified according to the behavioural results identified in the assay, or by measuring biomarkers of disease progression/treatment, such as IL-6, NF-kB, TNF-α, C-reactive protein, and any other biomarker known to be or potentially implicated in the development of the disease or disorder being studied. Those skilled in the art are familiar with the wide variety of animal models available for further testing the products of the invention.

In summary, Examples 1-3 above provide evidence from bioinformatics, from in vitro assays and from in vivo mammalian experimentation, respectively, that the combinations of the invention have surprising and unexpected effects from which therapeutic utility, including synergistic therapeutic effects, are determined. Based on these discoveries, the inventors have further refined their analysis to provide preferred embodiments of the formulations more generally disclosed above.

### Example 4: Unit Dosage Form (UDF) Oral Capsule Embodiments

While the invention covers all oral formulations described herein, specific attention is drawn to the combination of Boswellia extract with those cannabinoids in the amount set out in Table 1a, 1b and 1c. Further preferred combinations include but are not limited to: a fixed dose Boswellia extract+CBD:CBG, Boswellia extract+THCA:CBD, Boswellia extract+THC:CBD and Boswellia extract+THC:CBDA, Boswellia extract+THC:CBD:CBC. Among other things, these combinations are indicated for the treatment of joint pain and swelling associated with osteoarthritis of the knee. These preferred embodiments employ pharmaceutical oral softgel capsule formulations comprising fixed dose active pharmaceutical ingredients listed in Tables 3 and Examples 4-1 to 4-12. Treatment with such combinations has analgesic and anti-inflammatory effects without the recognized adverse side effects associated with NSAID use.

### Unit Dosage Form (UDF) Oral Capsule Embodiments

Preferred Therapeutic Indications: Pain and Inflammation.
Boswellia extract: Liquified resin comprising 40% AKBA w/w.
Cannabinoid: liquid extract.

### Example 4-1—TimeWarp A3 Capsule (SoftGel; Low-Dose; 350 mg)

| Ingredient | Component | Final |
|---|---|---|
| Liquid extract cannabinoid | | 18.2 mg |
| (55% w/w) | | |
| THCA | 9 mg | |
| THC | 1 mg | |
| Boswellia extract (40% AKBA) | 100 mg | 250 mg |
| Carrier oil having Omega-3 to | | 31.8 mg |
| Omega-6 ratio at least 1.0 | | |
| Net weight of capsule contents | | 300 mg |
| Capsule (SoftGel—vegetarian | | 49 mg |
| corresponding to Size 3) | | |
| Gross weight of capsule | | 349 mg |
| (estimated) | | |

### Example 4-2—Island Mist/TimeWarp A3 Capsule (SoftGel; Low-Dose; 430 mg)

| Ingredient | Component | Final |
|---|---|---|
| Liquid extract cannabinoid | | 36.4 mg |
| (55% w/w) | | |
| CBD | 10 mg | |
| THC | 10 mg | |
| Boswellia extract (40% AKBA) | 100 mg | 250 mg |
| Carrier oil having Omega-3 to | | 83.6 mg |
| Omega-6 ratio at least 1.0 | | |
| Net weight of capsule contents | | 370 mg |
| Capsule (SoftGel—vegetarian | | 62 mg |
| corresponding to Size 2) | | |
| Gross weight of capsule | | 432 mg |
| (estimated) | | |

### Example 4-3—TimeWarp A3 Capsule (Softgel; Low-Dose; 577 mg)

| Ingredient | Component | Final |
|---|---|---|
| Liquid extract cannabinoid | | 18.2 mg |
| (55% w/w) | | |
| THC | 10 mg | |
| Boswellia extract (40% AKBA) | 180 mg | 450 mg |
| Carrier oil having Omega-3 to | | 31.8 mg |
| Omega-6 ratio at least 1.0 | | |
| Net weight of capsule contents | | 500 mg |
| Capsule (SoftGel—vegetarian | | 77 mg |
| corresponding to Size 1) | | |
| Gross weight of capsule | | |
| (estimated) | | 577 mg |

### Example 4-4—Island Mist/TimeWarp A3 Capsule (SoftGel; Low-Dose THC/CBD; High Dose Boswellia Extract; 777 mg).

| Ingredient | Component | Final |
|---|---|---|
| Liquid extract cannabinoid | | 36.4 mg |
| (55% w/w) | | |
| CBD | 10 mg | |
| THC | 10 mg | |
| Boswellia extract (40% AKBA) | 200 mg | 500 mg |
| Carrier oil having Omega-3 to | | 143.6 mg |
| Omega-6 ratio at least 1.0 | | |
| Net weight of capsule contents | | 680 mg |
| Capsule (SoftGel—vegetarian | | 97 mg |
| corresponding to Size 0) | | |
| Gross weight of capsule | | 777 mg |
| (estimated) | | |

### Example 4-5—Purple x Chemo Extract Capsule (4:1 THC/CBG; High Dose AKBA Soft-Gel Capsule; 558 mg)

| Ingredient | | Final |
|---|---|---|
| Cannabis extract | | 33.3 mg |
| (60% cannabinoid in carrier) | | |
| THC | 10 mg | |
| CBG | 3 mg | |
| Boswelha extract (40% AKBA) | 100 mg | 250 mg |
| Carrier oil having Omega-3 to | | 76.6 mg |
| Omega-6 ratio at least 1.0 | | |
| Net weight of capsule contents | | 510 mg |
| Capsule (HardGel Size 1) | | 48 mg |
| Gross weight of capsule | | 558 mg |
| (estimated) | | |

### Example 4-6—1:1 THC/CBD; Low Dose AKBA Soft-Gel Capsule; 350 mg

| Ingredient | | Final |
|---|---|---|
| Cannabis extract | | 33.3 mg |
| (60% cannabinoid in carrier) | | |
| THC | 10 mg | |
| CBD | 10 mg | |
| Boswelha extract (40% AKBA) | 40 mg | 100 mg |
| Carrier oil having Omega-3 to | | 166.7 mg |
| Omega-6 ratio at least 1.0 | | |
| Net weight of capsule contents | | 300 mg |
| Capsule (Soft-gel) | | 50 mg |
| Gross weight of capsule | | 350 mg |

### Example 4-7—1:10 THC/CBD; Low Dose AKBA Soft-Gel Capsule; 350 mg

| Ingredient | | Final |
|---|---|---|
| Cannabis extract | | 18.3 mg |
| (60% cannabinoid in carrier) | | |
| THC | 1 mg | |
| CBD | 10 mg | |
| Boswelha extract (40% AKBA) | 40 mg | 100 mg |
| Carrier oil having Omega-3 to | | 131.7 mg |
| Omega-6 ratio at least 1.0 | | |
| Labrasol^{TM} surfactant | | 25 mg |
| Net weight of capsule contents as | | 300 mg |
| formulated | | |
| Capsule (Soft-gel) | | 50 mg |
| Gross weight of capsule | | 350 mg |

### Example 4-8—10:2:1 THC/CBG/CBC; High Dose AKBA Soft-Gel Capsule; 450 mg Volume

| Ingredient | | Final |
|---|---|---|
| Cannabis extract | | 21.7 mg |
| (60% w/w) | | |
| THC | 10 mg | |
| CBG | 2 mg | |
| CBC | 1 mg | |
| Other cannabis extract ingredients | | |
| included in liquid extract: residual | | |
| solvent, lipids, waxes, sugars, and | 8.7 mg | |
| other phytochemicals and | | |
| antioxidants | | |
| Boswellia extract (40% AKBA) | 100 mg | 250 mg |
| Carrier oil having Omega-3 to | | 178.3 mg |
| Omega-6 ratio at least 1.0 | | |
| Net weight of capsule contents | | 450 mg |
| Capsule (SoftGel Size 1) | | 77 mg |
| Gross weight of capsule | | 527 mg |
| (estimated) | | |

### Example 4-9—2:1:1 THC/CBG/CBC; High Dose AKBA Soft-Gel Capsule; 450 mg Volume

| Ingredient | | Final |
|---|---|---|
| Cannabis extract | | 20 mg |
| (60% w/w) | | |
| THC | 6 mg | |
| CBG | 3 mg | |
| CBC | 3 mg | |
| Other cannabis extract ingredients | 8 mg | |
| included in liquid extract: residual | | |
| solvent, lipids, waxes, sugars, and | | |
| other phytochemicals and | | |
| antioxidants | | |
| Boweilia extract (40% AKBA) | 100 mg | 250 mg |
| Carrier oil having Omega-3 to | | 180 mg |
| Omega-6 ratio at least 1.0 | | |
| Net weight of capsule contents | | 450 mg |
| Capsule (SoftGel Size 1) | | 77 mg |
| Gross weight of capsule | | 527 mg |
| (estimated) | | |

### Example 4-10—2:1:1 CBD/CBG/CBC; High Dose AKBA Soft-Gel Capsule; 450 mg Volume

| Ingredient | | Final |
|---|---|---|
| Cannabis extract | | 20 mg |
| (60% w/w) | | |
| CBD | 6 mg | |
| CBG | 3 mg | |
| CBC | 3 mg | |
| Other cannabis extract ingredients | 8 mg | |
| included in liquid extract: residual | | |
| solvent, lipids, waxes, sugars, and | | |
| other phytochemicals and | | |
| antioxidants | | |
| Bowelha extract (40% AKBA) | 100 mg | 250 mg |
| Carrier oil having Omega-3 to | | 180 mg |
| Omega-6 ratio at least 1.0 | | |
| Net weight of capsule contents | | 450 mg |
| Capsule (SoftGel Size 1) | | 77 mg |
| Gross weight of capsule | | 527 mg |
| (estimated) | | |

### Examples 4-11, 4-12, 4-13: Self-Microemulsifying (SMEDDS) Formulations

| | | Example | Example | Example |
|---|---|---|---|---|
| | | 4-8 | 4-9 | 4-10 |
| Description | Function | mg/cap | mg/cap | mg/cap |
| Boswellia Extract | Active | 25 | 50 | 100 |
| (liquid) | | | | |
| Oil having Omega-3 | Carrier Oil | 322.5 | 295 | 240 |
| to Omega-6 ratio | | | | |
| at least 1.0 | | | | |
| Labrasol ALF | Solubilizer/ | 150 | 150 | 150 |
| | Surfactant | | | |
| THC resin or | | 2.5 | 5 | 10 |
| distillate | Active | | | |

| | |
|---|---|
| Total Weight (mg) | 500 mg |

While the invention covers all oral formulations described herein, specific attention is drawn to the combination of Boswellia extract (40% AKBA) with those cannabinoids in the amount set out in Table 1A, 1B and 1C. Further preferred combinations include but are not limited to: a fixed dose Boswellia extract+CBD:CBG combination for the treatment of neuropathic pain; combinations of Boswellia extract+THCA:CBD, Boswellia extract+THC:CBD and Boswellia extract+THC:CBDA for treatment of chronic pain.

Oral softgel capsule formulations comprising fixed dose active pharmaceutical ingredients listed in the examples are especially preferred. Treatment with such combinations provides analgesic and anti-inflammatory effects without the recognized adverse side effects associated with NSAID use.

Further combinations of the invention are provided in Table 5

TABLE 5
Preferred Defined Dose combination products of the invention

| | | | Defined Dose (mg) | | | | | | | | |
|---|---|---|---|---|---|---|---|---|---|---|---|
| | | | Boswellia | | | | | | | Final | |
| | | | extract | | | | | | | capsule | |
| Prod | | Capsule | (40% | | | | | | | mass* | |
| # | Capsule | Fill | AKBA) | THC | THCA | THCV | CBC | CBD | CBG | (mg) | Indication |
| 4-1 | HardGel | Oil with | 100 | 1 | 9 | | | | | 349 | Pain, Nausea, |
| | | Omega-3 to | | | | | | | | | Obesity, Metabolic |
| | | Omega-6 | | | | | | | | | Syndrome, |
| | | ratio of at | | | | | | | | | Inflammation, anti |
| | | least 1.0 | | | | | | | | | cell proliferation |
| 4-2 | HardGel | Oil with | 100 | 10 | | | | 10 | | 432 | Pain, Appetite |
| | | Omega-3 to | | | | | | | | | enhancement, |
| | | Omega-6 | | | | | | | | | Inflammation; |
| | | ratio of at | | | | | | | | | |
| | | least 1.0 | | | | | | | | | |
| 4-3 | HardGel | Oil with | 180 | 10 | | | | | | 577 | Pain; Anxiety; |
| | | Omega-3 to | | | | | | | | | Sleep, |
| | | Omega-6 | | | | | | | | | Inflammation; |
| | | ratio of at | | | | | | | | | |
| | | least 1.0 | | | | | | | | | |
| 4-4 | HardGel | Oil with | 200 | 10 | | | | 10 | | 777 | Pain; Anxiety; |
| | | Omega-3 to | | | | | | | | | Sleep, |
| | | Omega-6 | | | | | | | | | Inflammation; |
| | | ratio of at | | | | | | | | | |
| | | least 1.0 | | | | | | | | | |
| 4-5 | HardGel | Oil with | 100 | 10 | | | | | 3 | 578 | Pain; Nausea, |
| | | Omega-3 to | | | | | | | | | Inflammation, |
| | | Omega-6 | | | | | | | | | Appetite |
| | | ratio of at | | | | | | | | | stimulation |
| | | least 1.0 | | | | | | | | | |
| 4-6 | HardGel | Oil with | 40 | 10 | | | | 10 | | 350 | Anti-epileptic, |
| | | Omega-3 to | | | | | | | | | Chronic Pain, |
| | | Omega-6 | | | | | | | | | Inflammation, |
| | | ratio of at | | | | | | | | | Schizophrenia, |
| | | least 1.0 | | | | | | | | | Diabetes |
| 4-7 | HardGel | Oil with | 40 | 1 | | | | 10 | | 350 | Pain; Nausea, |
| | | Omega-3 to | | | | | | | | | Inflammation, |
| | | Omega-6 | | | | | | | | | Appetite |
| | | ratio of at | | | | | | | | | stimulation |
| | | least 1.0 | | | | | | | | | |
| | | PLUS | | | | | | | | | |
| | | surfactant | | | | | | | | | |
| 4-8 | SoftGel | Surfactant | 25 | 2.5 | | | | | | 500 | Anti-epileptic, |
| | | (i.e, | | | | | | | | | Chronic Pain, |
| | | Labrasol ™) | | | | | | | | | Inflammation, |
| | | plus Oil | | | | | | | | | Schizophrenia; |
| | | with | | | | | | | | | Diabetes |
| | | Omega-3 to | | | | | | | | | |
| | | Omega-6 | | | | | | | | | |
| | | ratio of at | | | | | | | | | |
| | | least 1.0 | | | | | | | | | |
| 4-9 | SoftGel | Surfactant | 50 | 5.0 | | | | | | 500 | Anti-epileptic, |
| | | (i.e. | | | | | | | | | Chronic Pain, |
| | | Labrasol ™) | | | | | | | | | Inflammation, |
| | | plus Oil | | | | | | | | | Schizophrenia; |
| | | with | | | | | | | | | Diabetes |
| | | Omega-3 to | | | | | | | | | |
| | | Omega-6 | | | | | | | | | |
| | | ratio of at | | | | | | | | | |
| | | least 1.0 | | | | | | | | | |
| 4-10 | SoftGel | Surfactant | 100 | 10.0 | | | | | | 500 | Anti-epileptic, |
| | | (i.e, | | | | | | | | | Chronic Pain, |
| | | Labrasol ™) | | | | | | | | | Inflammation, |
| | | plus Oil | | | | | | | | | Schizophrenia; |
| | | with | | | | | | | | | Diabetes |
| | | Omega-3 to | | | | | | | | | |
| | | Omega-6 | | | | | | | | | |
| | | ratio of at | | | | | | | | | |
| | | least 1.0 | | | | | | | | | |
| 4-11 | SoftGel | Oil with | 80 | 10 | | | | 10 | | 350 | Pain; Anxiety; |
| | | Omega-3 to | | | | | | | | | Sleep, |
| | | Omega-6 | | | | | | | | | Inflammation |
| | | ratio of at | | | | | | | | | |
| | | least 1.0 | | | | | | | | | |
| 4-12 | SoftGel | Oil with | 80 | 1 | | | | 10 | | 300 | Anti-epileptic, |
| | | Omega-3 to | | | | | | | | | Chronic Pain, |
| | | Omega-6 | | | | | | | | | Inflammation, |
| | | ratio of at | | | | | | | | | Schizophrenia; |
| | | least 1.0 | | | | | | | | | Diabetes |
| 4-13 | SoftGel | Surfactant | 100 | 1 | | | | 10 | | 300 | Anti-epileptic, |
| | | (i.e, | | | | | | | | | Chronic Pain, |
| | | Labrasol ™) | | | | | | | | | Inflammation, |
| | | plus Oil | | | | | | | | | Schizophrenia; |
| | | with | | | | | | | | | Diabetes |
| | | Omega-3 to | | | | | | | | | |
| | | Omega-6 | | | | | | | | | |
| | | ratio of at | | | | | | | | | |
| | | least 1.0 | | | | | | | | | |
| 4-14 | SoftGel | Oil with | 40 | | | 10 | | 10 | | 548 | Pain; Anxiety; |
| | | Omega-3 to | | | | | | | | | Sleep, |
| | | Omega-6 | | | | | | | | | Inflammation; |
| | | ratio of at | | | | | | | | | Diabetes; Appetite |
| | | least 1.0 | | | | | | | | | supression |
| 4-15 | HardGel | Surfactant | 100 | 5 | | | | 10 | | 548 | Anti-epileptic, |
| | | (i.e. | | | | | | | | | Chronic Pain, |
| | | Labrasol ™) | | | | | | | | | Inflammation, |
| | | plus Oil | | | | | | | | | Schizophrenia, |
| | | with | | | | | | | | | Diabetes |
| | | Omega-3 to | | | | | | | | | |
| | | Omega-6 | | | | | | | | | |
| | | ratio of at | | | | | | | | | |
| | | least 1.0 | | | | | | | | | |
| 4-16 | SoftGel | Surfactant | 200 | 5 | | | | 10 | | 250 | Anti-epileptic, |
| | | (i.e. | | | | | | | | | Chronic Pain, |
| | | Labrasol ™) | | | | | | | | | Inflammation, |
| | | plus Oil | | | | | | | | | Schizophrenia, |
| | | with | | | | | | | | | Diabetes |
| | | Omega-3 to | | | | | | | | | |
| | | Omega-6 | | | | | | | | | |
| | | ratio of at | | | | | | | | | |
| | | least 1.0 | | | | | | | | | |
| 4-17 | SoftGel | Surfactant | 200 | 10 | | | 10 | 10 | | 250 | Anti-bacterial, |
| | | (i.e. | | | | | | | | | AntiFungal, |
| | | Labrasol ™) | | | | | | | | | Chronic Pain, |
| | | plus Oil | | | | | | | | | Chronic Stress, |
| | | with | | | | | | | | | Depression, |
| | | Omega-3 to | | | | | | | | | Inflammation |
| | | Omega-6 | | | | | | | | | |
| | | ratio of at | | | | | | | | | |
| | | least 1.0 | | | | | | | | | |

*including capsule shell and all carrier, filler, stabilizer, and anti-oxidant, etc.

Any of the capsules provided herein may include an extended release (enteric) coating. An example of a suitable enteric coating is provided in Table 6.

TABLE 6
Optional extended release coating for use with capsules of the invention.

| Component | Function | % w/w | mg/capsule |
|---|---|---|---|
| Ethylcellulose | Water-insoluble | 71.4 | 67 |
| dispersion | film-forming | | |
| (Aquacoat ECD 30) | polymer | | |
| Triethyl citrate | Plasticizer | 14.3 | 13 |
| Polyvinyl alcohol/ | Water-soluble | 14.3 | 13 |
| polyethylene | pore former | | |
| glycol co-polymer | | | |
| (Kollicoat IR) | | | |
| Total | | 100.0% | 93 mg |

While preferred embodiments of the present invention have been shown and described herein, those skilled in the art recognize that such embodiments are provided by way of example only. Numerous variations, changes, and substitutions will occur to those skilled in the art without departing from the invention. It should be understood that various alternatives to the embodiments of the invention described herein may be employed in practicing the invention. It is intended that the following claims define the scope of the invention and that methods and formulations within the scope of these claims and their equivalents be covered thereby.

What is claimed is:

1. An oral formulation comprising
a. one or more cannabinoids selected from among the group consisting of:
0.1-750 mg tetrahydrocannabinolic acid (THCA),
0.1-100 mg tetrahydrocannabinol (THC),
0.1-750 mg cannabidiolic acid (CBDA),
0.1-750 mg cannabidiol (CBD), 0.1-750 mg cannabichromene (CBC), and
0.1-750 mg cannabigerol (CBG); and
b. a Boswellia extract.

2. The oral formulation of claim 1 in a unit dosage form selected from the group consisting of a pill, tablet, capsule, film, wafer, lollipop, lozenge, oil, tincture, and syrup.

3. The oral formulation of claim 2, wherein the formulation is an orally disintegrating film, or wafer.

4. The oral formulation of claim 1 or 2, wherein the formulation is a pill or tablet and further comprises an enteric coating for containing the one or more cannabinoids and the lipid carrier.

5. The oral formulation of any one of claims 1, 2 or 4, wherein the formulation is a pill, tablet, or capsule, and further comprises an outer shell that is substantially opaque to one or both of ultraviolet and visible light.

6. The oral formulation of any one of claims 1, 2, 4 or 5 further comprising a carrier oil.

7. The oral formulation of any one of claims 1 to 6 further comprising a surfactant.

8. The oral formulation of any one of claims 1 to 7, wherein one or more of the cannabinoids is present in the form of an organic solvent-based extract of Cannabis.

9. The oral formulation of any one of claims 1, 2 or 4 to 6, wherein one or more of the cannabinoids is present in the form of dried Cannabis flower.

10. The oral formulation of any one of claims 1 to 9, wherein one or more of the cannabinoids is the product of a biosynthetic process in yeast, a microbe, a non-Cannabis cell-based system or a cell-free system.

11. The oral formulation of any one of claims 1 to 10, wherein the formulation comprises a defined dose or combination dose of cannabinoid(s) selected from the list consisting of (each cannabinoid milligram amount about or equal to):
THC (10 mg), CBD (10 mg), and Boswellia extract (100 mg),
THC (10 mg), CBG (3 mg), and Boswellia extract (100 mg),
THC (1 mg), CBD (25 mg), and Boswellia extract (100 mg),
THC (10 mg), CBD (10 mg), and Boswellia extract (100 mg),
THC (5 mg), CBD (20 mg), and Boswellia extract (100 mg),
THC (10 mg), CBG (3 mg), and Boswellia extract (100 mg),
THC (1 mg), CBD (10 mg), and Boswellia extract (100 mg),
THC (1 mg), THCA (9 mg), and Boswellia extract (100 mg),
THC (10 mg), CBD (10 mg), and Boswellia extract (100 mg),
THC (10 mg) and Boswellia extract (180 mg),
THC (10 mg), CBD (10 mg), and Boswellia extract (200 mg),
THC (10 mg), CBG (3 mg), and Boswellia extract (100 mg),
THC (10 mg), CBD (10 mg), and Boswellia extract (40 mg),
THC (1 mg), CBD (10 mg), and Boswellia extract (40 mg),
THC (2.5 mg) and Boswellia extract (25 mg),
THC (5 mg) and Boswellia extract (50 mg),
THC (10 mg) and Boswellia extract (100 mg),
THC (10 mg), CBD (10 mg), and Boswellia extract (80 mg),
THC (1 mg), CBD (10 mg), and Boswellia extract (80 mg),
THC (1 mg), CBD (10 mg), and Boswellia extract (100 mg),
THCV (10 mg), CBD (10 mg), and Boswellia extract (40 mg),
THC (5 mg), CBD (10 mg), and Boswellia extract (100 mg),
THC (1 mg), CBD (10 mg), and Boswellia extract (200 mg),
THC (10 mg), CBC (10 mg), CBD (10 mg), and Boswellia extract (40 mg),
THC (10 mg), CBG (3 mg), CBC (3 mg) and Boswellia extract (100 mg),
THC (10 mg), CBG (10 mg), CBC (10mg) and Boswellia extract (200 mg), and
THC (10 mg), CBG (3 mg), CBC (3 mg) and Boswellia extract (200 mg).

12. The oral formulation of any one of claims 1 to 11, further comprising at least one further cannabinoid selected from the group consisting of CBN, CBG, CBGA, CBC, and THCV.

13. The oral formulation of any one of claims 1 to 12, comprising CBD in an amount between 10-50 mg.

14. The oral formulation of claim 13, comprising 25 mg CBD.

15. The oral formulation of any one of claims 1 to 14, comprising 500 mg CBD.

16. The oral formulation of any one of claims 1 to 15, wherein the Boswellia extract comprises about 5-20% of the oral formulation by weight.

17. The oral formulation of claim 16, wherein the Boswellia extract is present in an amount of about 25 mg to 1000 mg, or about 50 mg, about 100 mg, or about 500 mg.

18. The oral formulation of claim 16, wherein the Boswellia extract is an organic botanical solvent-based extract.

19. The oral formulation of any one of claims 1 to 18, wherein formulation comprises about 2% to about 20% by weight of AKBA or Boswellic acid, or a combination thereof.

20. The oral formulation of any one of claims 1 to 18, wherein the cannabinoid is physically separated from the Boswellia extract and/or the AKBA.

21. The oral formulation of any one of claims 1 to 18, wherein the cannabinoid is evenly dispersed within at least a portion of the oral formulation.

22. The oral formulation of any one of claims 1 to 21, wherein a signifier which signifies the cannabinoid dosage is associated directly with the oral formulation by embossing, or by colour, pattern or shape feature.

23. The oral formulation of claim 22, wherein the signifier is adapted to be directly interpreted by a consumer and/or is a machine-readable code.

24. The oral formulation of any one of claims 1 to 23, wherein the oral formulation is contained in an individual blister pack sealed in an inert gas atmosphere comprising little or no oxygen.

25. The oral formulation of any one of claims 1-2, 4-8 or 10-24, further comprising a softgel containing the one or more cannabinoids and/or the Boswellia extract in a liquid fill.

26. The oral formulation of claim 25, wherein the Boswellia extract comprises a carrier oil.

27. The oral formulation of claim 26 wherein the carrier oil comprises an oil having a lipid ratio by weight of Omega-3 to Omega-6 of 1 or higher.

28. The oral formulation of claim 27 wherein the liquid fill further comprises a surfactant.

29. The oral formulation of any one of claims 1 to 28, wherein the one or more cannabinoids, and/or the Boswellia extract, is present in a defined dose.

30. A method of administering a cannabinoid to an individual, the method comprising administering Boswellia extract to the individual in combination with the cannabinoid, in an oral formulation of any one of claims 1 to 29.

31. The method of claim 30, wherein the individual is suffering from one or more diseases, conditions or disorders selected from the group consisting of pain, inflammation, anxiety, depression, sleep disorders, insomnia, lack of energy, lack of alertness, weight gain, obesity, diabetes, Metabolic Syndrome, acute and anticipatory nausea, suppressed appetite, epilepsy, spasticity, schizophrenia, bi-polar disorder, cancer and neoplasia, chronic pain, osteoarthritic pain, bacterial and/or fungal infections and fibromyalgia.

32. The method of claim 31, wherein the administering results in amelioration and/or treatment of one or more symptoms selected from the group consisting of pain, inflammation, anxiety, depression, sleep disorders, insomnia, lack of energy, lack of alertness, weight gain, obesity, diabetes, Metabolic Syndrome, acute and anticipatory nausea, suppressed appetite, epilepsy, spasticity, schizophrenia, bi-polar disorder, cancer and neoplasia, chronic pain, osteoarthritic pain, bacterial and/or fungal infections and fibromyalgia.

33. A method of treating an individual suffering from one or more diseases, conditions or disorders selected from the group consisting of pain, inflammation, anxiety, depression, sleep disorders, insomnia, lack of energy, lack of alertness, weight gain, obesity, diabetes, Metabolic Syndrome, acute and anticipatory nausea, suppressed appetite, epilepsy, spasticity, schizophrenia, bi-polar disorder, cancer and neoplasia, chronic pain, osteoarthritic pain, bacterial and/or fungal infections, and fibromyalgia, the method comprising administration to the individual of a therapeutically effective amount of an oral formulation of any one of claims 1 to 29.

34. A method of manufacturing an oral formulation of any one of claims 1 to 29, comprising:
a. providing an organic extract of cannabinoids from cultivated Cannabis,
b. measuring the concentration of one or more cannabinoids selected from the group consisting of THCA, THC, CBDA, CBC, and CBD in the organic extract,
c. adjusting the concentration of one or more cannabinoids in the extract to prepare an adjusted extract within the defined dose tolerance limits of a manufacturing specification for the oral formulation; and
d. manufacturing the oral formulation with the adjusted extract.

35. A method of manufacturing an oral formulation of any one of claims 1 to 29, comprising
a. grinding a sample of Cannabis such that it passes through a mesh of average opening size of about 0.595 mm, about 0.250 mm, or about 0.125 mm to produce a ground sample,
b. measuring the concentration of one or more cannabinoids selected from the group consisting of THCA, THC, CBDA, CBC, and CBD in the ground sample,
c. adjusting the concentration of one or more cannabinoids in the ground sample to prepare an adjusted ground sample within the defined dose tolerance limits of a manufacturing specification for the oral formulation;
d. manufacturing the oral formulation with the adjusted ground sample.

36. Use of the oral formulation of any one of claims 1 to 28 for treating a disease or disorder in a subject in need thereof.

37. The use of claim 36, wherein the disease or disorder is selected from the group consisting of pain, inflammation, anxiety, depression, sleep disorders, insomnia, lack of energy, lack of alertness, weight gain, obesity, diabetes, Metabolic Syndrome, acute and anticipatory nausea, suppressed appetite, epilepsy, spasticity, schizophrenia, bi-polar disorder, cancer and neoplasia, chronic pain, osteoarthritic pain, bacterial and/or fungal infections and fibromyalgia.

38. A method of preparing an oral formulation, the method comprising:
a. preparing or obtaining a ground or milled Cannabis plant material preparation;
b. passing the Cannabis plant material preparation through a mesh or sieve to obtain a retained fraction of the material which is retained in the mesh or sieve and a pass-through fraction of the material which has passed through the mesh or sieve;
c. determining the content of the one or more cannabinoids of interest in the retained fraction and/or the pass-through fraction;
d. based on the content determined in step (c), determining a target amount of retained fraction material, pass-through material, or a combination thereof, to be incorporated into the formulation such that the formulation includes the desired defined dose of the one or more desired cannabinoids; and
e. combining the target amount of retained fraction material, pass-through material, or combination thereof with Boswellia extract, thereby forming the formulation.

39. The oral formulation of any one of claims 1 to 29, wherein the oral formulation comprises a ground or milled Cannabis plant material preparation which in turn comprises the one or more cannabinoids, wherein the preparation is prepared by a process comprising the following steps:
a. preparing or obtaining a ground or milled Cannabis plant material preparation;
b. passing the Cannabis plant material preparation through a mesh or sieve to obtain a retained fraction of the material which is retained in the mesh or sieve and a pass-through fraction of the material which has passed through the mesh or sieve;
c. determining the content of the one or more cannabinoids of interest in the retained fraction and/or the pass-through fraction; and
d. based on the content determined in step (c), determining a target amount of retained fraction material, pass-through material, or a combination thereof, to be incorporated into the formulation such that the formulation includes the desired defined dose of the one or more desired cannabinoids.
